SEQUENCE LISTING (34 sequences)

SEQ ID NO: 1 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacacaaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccactattaaataataatgctaattattctgaacttattccgtgtttatctttttataacgagtaa

SEQ ID NO: 2 - protein, 617 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYTNTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 3 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 4 - protein, 617 aa, Pasteurella multocida; identical to SEQ ID NO: 2

SEQ ID NO: 5 - DNA, 1956 bp, Pasteurella multocida
atgaagggaaaaaaagagatgactcaaattcaaatagctaaaaatccaccccaacatgaaaaagaaaatgaactcaacacctttcaaaataaaattgatagtctaaaaacaactttaaacaaagacattatttctcaacaaactctattggcaaaacaggacagtaaacatccgctatccgcatcccttgaaaacgaaaataaacttttattaaaacaactccaattggttctgcaagaatttaaaaaaatatatacctataatcaagcattagaagcaaagctagaaaaagataagcaaacaacatcaataacagatttatataatgaagtcgctaaaagtgatttagggttagtcaaagaaaccaacagcgcaaatccattagtcagtattatcatgacatctcacaatacagcgcaatttatcgaagcttctattaattcattattgttacaaacatataaaaacatagaaattattattgtagatgatgatagctcggataatacatttgaaattgcctcgagaatagcgaatacaacaagcaaagtcagagtatttagattaaattcaaacctaggaacttactttgcgaaaaatacaggcatattaaaatctaaaggtgacattattttctttcaagatagtgatgatgtatgtcatcatgaaagaatagaaagatgtgtaaatatattattagctaataaagaaactattgctgttcgttgtgcatactcaagactagcaccagaaacacagcatatcattaaagtcaataatatggattatagattaggttttataaccttgggtatgcacaaaaaagtatttcaagaaattggtttcttcaattgtacgactaaaggctcagatgatgagttttttcatagaattgcgaaatattatggaaaagaaaaaataaaaaatttactcttgccgttatactacaacacaatgagagaaaactctttatttactgatatggttgaatggatagacaatcataacataatacagaaaatgtctgataccagacaacattatgcaaccctgtttcaagcgatgcataacgaaactgcctcacatgatttcaaaaatctttttcaattccctcgtatttacgacgccttaccagtaccacaagaaatgagtaagttgtccaatcctaagattcctgtttatatcaatatttgttctattccctcaagaatagcgcaattacaacgtattatcggcatactaaaaaatcaatgtgatcattttcatatttatcttgatggctatgtagaaatccctgacttcataaaaaatttaggtaataaagcaaccgttgttcattgcaaagataaagataactccattagagataatggcaaattcattttactggaagagttgattgaaaaaaatcaagatggatattatataacctgtgatgatgacattatctatccaagcgattacatcaatacgatgatcaagaagctgaatgaatacgatgataaagcggttattggtttacacggcattctctttccaagtagaatgaccaaatatttttcggcggatagactggtatatagcttctataaacctctggaaaaagacaaagcggtcaatgtattaggtacaggaactgttagctttagagtcagtctctttaatcaattttctctttctgactttacccattcaggcatggctgatatctatttctctctcttgtgtaagaaaaataatattcttcagatttgtatttcaagaccagcaaactggctaacggaagataatagagacagcgaaacactctatcatcaatatcgagacaatgatgagcaacaaactcagctgatcatggaaaacggtccatggggatattcaagtatttatccattagtcaaaaatcatcctaaatttactgaccttatcccctgtttacctttttattttttataa

SEQ ID NO: 6 - protein, 651 aa, Pasteurella multocida
MKRKKEMTQIQIAKNPPQHEKENELNTFQNKIDSLKTTLNKDIISQQTLLAKQDSKHPLSASLENENKLLLKQLQLVLQEFKKIYTYNQALEAKLEKDKQTTSITDLYNEVAKSDLGLVKETNSANPLVSIIMTSHNTAQFIEASINSLLLQTYKNIEIIIVDDDSSDNTFEIASRIANTTSKVRVFRLNSNLGTYFAKNTGILKSKGDIIFFQDSDDVCHHERIERCVNILLANKETIAVRCAYSRLAPETQHIIKVNNMDYRLGFITLGMHKKVFQEIGFFNCTTKGSDDEFFHRIAKYYGKEKIKNLLLPLYYNTMRENSLFTDMVEWIDNHNIIQKMSDTRQHYATLFQAMHNETASHDFKNLFQFPRIYDALPVPQEMSKLSNPKIPVYINICSIPSRIAQLQRIIGILKNQCDHFHIYLDGYVEIPDFIKNLGNKATVVHCKDKDNSIRDNGKFILLEELIEKNQDGYYITCDDDIIYPSDYINTMIKKLNEYDDKAVIGLHGILFPSRMTKYFSADRLVYSFYKPLEKDKAVNVLGTGTVSFRVSLFNQFSLSDFTHSGMADIYFSLLCKKNNILQICISRPANWLTEDNRDSETLYHQYRDNDEQQTQLIMENGPWGYSSIYPLVKNHPKFTDLIPCLPFYFL

SEQ ID NO: 7 - protein, 238 aa, Escherichia coli
MIVANMSSYPPRKKELVHSIQSLHAQVDKINLCLNEFEEIPEELDGFSKLNPVIPDKDYKDVGKFIFPCAKNDMIVLTDDDIIYPPDYVEKMLNFYNSFAIFNCIVGIHGCIYIDAFDGDQSKRKVFSFTQGLLRPRVVNQLGTGTVFLKADQLPSLKYMDGSQRFVDVRFSRYMLENEIGMICVPREKNWLREVSSGSMEGLWNTFTKKWPLDIIKETQAIAGYSKLNLELVYNVEG

SEQ ID NO: 8 - protein, 520 aa, Escherichia coli
MNAEYINLVERKKKLGTNIGALDFLLSIHKEKVDLQHKNSPLKGNDNLIHKRINEYDNVLELSKNVSAQNSGNEFSYLLGYADSLRKVGMLDTYIKIVCYLTIQSRYFKNGERVKLFEHISNALRYSRSDFLINLIFERYIEYINHLKLSPKQKDFYFCTKFSKFHDYTKNGYKYLAFDNQADAGYGLTLLLNANDDMQDSYNLLPEQELFICNAVIDNMNIYRSQFNKCLRKYDLSEITDIYPNKIILQGIKFDKKKNVYGKDLVSIIMSVFNSEDTIAYSLHSLLNQTYENIEILVCDDCSSDKSLEIIKSIAYSSSRVKVYSSRKNQGPYNIRNELIKKAHGNFITFQDADDLSHPERIQRQVEVLRNNKAVICMANWIRVASNGKIQFFYDDKATRMSVVSSMIKKDIFATVGGYRQSLIGADTEFYETVIMRYGRESIVRLLQPLILGLWGDSGLTRNKGTEALPDGYISQSRREYSDIAARQRVLGKSIVSDKDVRGLLSRYGLFKDVSGIIEQ

SEQ ID NO: 9 - protein, 562 aa, Escherichia coli
MNKLVLVGHPGSKYQIVEHFLKEIGMNSPNYSTSNKISPEYITASLCQFYQTPEVNDVVDEREFSAVQVSTMWDSMVLELMMNNLNNKLWGWADPSIIFFLDFWKNIDKSIKFIMIYDHPKYNLMRSVNNAPLSLNINNSVDNWIAYNKRLLDFFLENKERCVLINFEAFQSNKKNIIKPLSNIIKIDNLMSAHYKNSILFDVVENNDYTKSNEIALLEKYTTLFSLSANETEITFNDTKVSEYLVSELIKERTEVLKLYNELQAYANLPYIETSKDNVSAEAALWEVVEERNSIFNIVSHLVQESKKKDADIELTKSIFKKRQFLLLNRINELKKEKEEVIKLSKINHNDVVRQEKYPDDIEKKINDIQKYEEEISEKESKLTQAISEKEQILKQLHKYEEEISEKESKLTQAISEKEQILKQLHIVQEQLEHYFIENQEIKKKLPPVLYGAAEQIKQELGYRLGYIIVSYSKSLKGIITMPFALIRECVFEKKRKKSYGVDVPLYLYADADKAERVKKHLSYQLGQAIISSANSIFGFITLPFKLIVVVYKYRRAKIKGC

SEQ ID NO: 10 - protein, 150 aa, Pasteurella multocida
APPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYR

SEQ ID NO: 11 - protein, 99 aa, Pasteurella multocida
YITCDDDIRYPADYINTMIKKINKYNDKAAIGLHGVIFPSRVNKYFSSDRIVYNFQKTFRKDTAVNILGTGTVAFRVSIFNKFSLSDFEHPGMVDIYFS

SEQ ID NO: 12 - DNA, 1722 bp, Pasteurella multocida
atgaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 13 - protein, 573 aa, Pasteurella multocida
MNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 14 - DNA, 1626 bp, Pasteurella multocida
ataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 15 - protein, 541 aa, Pasteurella multocida
MSNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 16 - DNA, 34 bp, Artificial Sequence
  sense primer
atgaatataacacaatcaaaaagtaataaaatag

SEQ ID NO: 17 - DNA, 31 bp, Artificial sequence
  sense primer
atgagcaatagtgaattagggattacaaaag

SEQ ID NO: 18 - DNA, 28 bp, Artificial sequence
  sense primer
atgagcttatttaaacgtgctactgagc

SEQ ID NO: 19 - DNA, 34 bp, Artificial Sequence
  anti-sense primer
tttactcgttataaaaagataaacacggaataag

SEQ ID NO: 20 - DNA, 25 bp, Artificial Sequence
  sense primer
atgaagagaaaaaaagagatgactc

SEQ ID NO: 21 - DNA, 30 bp, Artificial Sequence
  anti-sense primer
atcattataaaaaataaaaaggtaaacagg

SEQ ID NO: 22 - protein, 78 aa, Artificial Sequence
  Motif I
QTYXNXEXXXXDDXXXDXXXXXXXXXIAXXXXXXVXXXXXXXNXGXYXXXXXXXXXXXXXXXXFQDXDDXXHXERIXR

SEQ ID NO: 23 - protein, 82 aa, Artificial Sequence
  Motif II
XDXGKFIXXXXXXXXXXXXDDDIXYPXDYXXXMXXXXXXXXXXXXXXXXXXXXXXXXXXXXXXXXXXXXXXXXVNXLGTGTV

SEQ ID NO: 24 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcagaatagcaatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 25 - protein, 617 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQNSNDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 26 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtaatgataatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 27 - protein, 617 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCNDNIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 28 - DNA, 42 bp, Artificial Sequence
  sense primer
atattattttctttcagaatagcaatgatgtatgtcaccatg

SEQ ID NO: 29 - DNA, 42 bp, Artificial Sequence
  antisense primer
catggtgacatacatcattgctattctgaaagaaaataatat

SEQ ID NO: 30 - DNA, 37 bp, Artificial Sequence
  sense primer
gatattatataacttgtaatgataatatccggtatcc

SEQ ID NO: 31 - DNA, 37 bp, Artificial Sequence
  antisense primer
ggataccggatattatcattacaagttatataatatc

SEQ ID NO: 32 - protein, 21 aa, artificial sequence
  synthetic peptide
KGDIIFFQDSDDVCHHERIER

SEQ ID NO: 33 - DNA, 1956 bp, Pasteurella multocida
atgaagggaaaaaaagagatgactcaaattcaaatagctaaaaatccaccccaacatgaaaaagaaaatgaactcaacacctttcaaaataaaattgatagtctaaaaacaactttaaacaaagacatcatttctcaacaaactttattggcaaaacaggacagtaaacatccgctatccgcatcccttgaaaacgaaaataaacttttattaaaacaactccaattggttctgcaagaatttgaaaaaatatatacctataatcaagcattagaagcaaagctagaaaaagataagcaaacaacatcaataacagatttatataatgaagtcgctaaaagtgatttagggttagtcaaagaaactaacagcgcaaatccattagtcagtattatcatgacatctcacaatacagcgcaatttatcgaagcttctattaattcattattgttacaaacatataaaaacatagaaattattattgtagatgatgatagctcggataatacatttgaaattgcctcgagaatagcgaatacgacaagcaaagtcagagtatttagattaaattcaaacctaggaacttactttgcgaaaaatacaggcatattaaaatctaaaggtgacattattttctttcaagatagtgatgatgtatgtcatcatgaaagaatagaaagatgtgtaaatatattattagctaataaagaaactattgctgttcgttgtgcatactcaagactagcaccagaaacacaacatatcattaaagtcaataatatggattatagattaggttttataaccttgggtatgcacagaaaagtatttcaagaaattggtttcttcaattgtacgactaaaggctcagatgatgagttttttcatagaattgcgaaatattatggaaaagaaaaaataaaaaatttactcttgccgttatactacaacacaatgagagaaaactctttatttactgatatggttgaatggatagacaatcataacataatacagaaaatgtctgataccagacaacattatgcaaccctgtttcaagcgatgcataacgaaactgcctcacatgatttcaaaaatctttttcaattccctcgtatttacgatgccttaccagtaccacaagaaatgagtaagttgtccaatcctaagattcctgtttatatcaatatttgttctattccctcaagaatagcgcaattacgacgtattatcggcatactaaaaaatcaatgtgatcattttcatatttatcttgatggctatgtagaaatccctgacttcataaaaaatttaggtaataaagcaaccgttgttcattgcaaagataaagataactccattagagataatggcaaattcattttactggaagagttgattgaaaaaaatcaagatggatattatataacctgtgatgatgacattatctatccaagcgattacatcaatacgatgatcaagaagctgaatgaatacgatgataaagcggttattggtttacacggcattctctttccaagtagaatgaccaaatatttttcggcggatagactggtatatagcttctataaacctctggaaaaagacaaagcggtcaatgtattaggtacaggaactgttagctttagagtcagtctctttaatcaattttctctttctgactttacccattcaggcatggctgatatctatttctctctcttgtgtaagaaaaataatattcttcagatttgtatttcaagaccagcaaactggctaacagaagataatagagacagcgaaacactctatcatcaatatcgagacaatgatgagcaacaaactcagctgatcatggaaaacggtccatggggatattcaagtatttatccattagtcaaaaatcatcctaaatttactgaccttatcccctgtttacctttttattttttataa

SEQ ID NO: 34 - protein, 651 aa, Pasteurella multocida
MKRKKEMTQIQIAKNPPQHEKENELNTFQNKIDSLKTTLNKDIISQQTLLAKQDSKHPLSASLENENKLLLKQLQLVLQEFEKIYTYNQALEAKLEKDKQTTSITDLYNEVAKSDLGLVKETNSANPLVSIIMTSHNTAQFIEASINSLLLQTYKNIEIIIVDDDSSDNTFEIASRIANTTSKVRVFRLNSNLGTYFAKNTGILKSKGDIIFFQDSDDVCHHERIERCVNILLANKETIAVRCAYSRLAPETQHIIKVNNMDYRLGFITLGMHRKVFQEIGFFNCTTKGSDDEFFHRIAKYYGKEKIKNLLLPLYYNTMRENSLFTDMVEWIDNHNIIQKMSDTRQHYATLFQAMHNETASHDFKNLFQFPRIYDALPVPQEMSKLSNPKIPVYINICSIPSRIAQLRRIIGILKNQCDHFHIYLDGYVEIPDFIKNLGNKATVVHCKDKDNSIRDNGKFILLEELIEKNQDGYYITCDDDIIYPSDYINTMIKKLNEYDDKAVIGLHGILFPSRMTKYFSADRLVYSFYKPLEKDKAVNVLGTGTVSFRVSLFNQFSLSDFTHSGMADIYFSLLCKKNNILQICISRPANWLTEDNRDSETLYHQYRDNDEQQTQLIMENGPWGYSSIYPLVKNHPKFTDLIPCLPFYFL

(19) United States
(12) Patent Application Publication
(10) Pub. No.: US 2004/0197868 A1
(43) Pub. Date: Oct. 7, 2004

(54) Heparin/heparosan synthase from P. multocida, soluble and single action catalysts thereof and methods of making and using same

(76) Inventor: Paul L. DeAngelis, Edmond, OK (US)

Correspondence Address: DUNLAP, CODDING & ROGERS P.C., PO BOX 16370, OKLAHOMA CITY, OK, 73113, US

(21) Appl. No.: 10/814,752

(22) Filed: Mar. 31, 2004

Related U.S. Application Data
(63) Continuation-in-part of application No. 10/142,143, filed on May 8, 2002.
(60) Provisional application No. 60/458,939, filed on Mar. 31, 2003.
(60) Provisional application No. 60/289,554, filed on May 8, 2001.
(60) Provisional application No. 60/296,386, filed on Jun. 6, 2001.
(60) Provisional application No. 60/303,691, filed on Jul. 6, 2001.
(60) Provisional application No. 60/313,258, filed on Aug. 17, 2001.

(51) Int. Cl. C12N 9/24; C07H 21/04

(52) U.S. Cl. 435/69.1; 435/200; 435/320.1; 435/325; 536/23.2

(57) ABSTRACT

The presently claimed and disclosed invention relates, in general, to single action, dual action and soluble heparin synthases and, more particularly, to single action, dual action and soluble heparin synthases obtained from Pasteurella multocida. The presently claimed and disclosed invention also relates to heparosan, heparin and heparin-like molecules provided by recombinant techniques and methods of using such molecules. The presently claimed and disclosed invention also relates to methods, and molecules produced according to such methods, for using the presently claimed and disclosed heparosan and/or heparin synthase for polymer grafting and the production of non-naturally occurring chimeric polymers incorporating stretches of one or more acidic GAG molecules, such as heparin, chondroitin, hyaluronan, and/or heparosan.

### CROSS-REFERENCE TO RELATED APPLICATIONS

This application claims priority under 35 U.S.C. § 119(e) of U.S. Provisional Application Serial No. 60/458,939, filed Mar. 31, 2003, entitled “IDENTIFICATION OF NEW HEPAROSAN SYNTHASES AND CREATION OF SOLUBLE CATALYSTS”, the contents of which are hereby expressly incorporated herein by reference in their entirety. This application is also a continuation-in-part of U.S. Ser. No. 10/142,143, filed May 8, 2002, entitled “HEPARIN/HEPAROSAN SYNTHASE FROM P. MULTOCIDA AND METHODS OF MAKING AND USING SAME”; which claims priority under 35 U.S.C. § 119(e) of U.S. Provisional Applications Serial No. 60/289,554, filed May 8, 2001, entitled “PASTEURELLA MULTOCIDA HEPARIN SYNTHASE GENE AND METHODS OF MAKING AND USING SAME”; Serial No. 60/296,386, filed Jun. 6, 2001, entitled “HEPARIN AND HEPARIN-LIKE POLYSACCHARIDES, THEIR SYNTHASES, AND USES THEREOF”; Serial No. 60/303,691, filed Jul. 6, 2001, entitled “ENABLEMENT OF RECOMBINANT HEPARIN SYNTHASE, pmHAS”; and Serial No. 60/313,258, filed Aug. 17, 2001, entitled “HEPARIN SYNTHASE SEQUENCE MOTIFS AND METHODS OF MAKING AND USING SAME”, the contents of which are hereby expressly incorporated in their entirety by reference.

### STATEMENT REGARDING FEDERALLY FUNDED RESEARCH

The government owns certain rights in and to this application pursuant to a grant from the National Science Foundation (NSF), Grant No. MCB-9876193.

### BACKGROUND OF THE INVENTION

1. Field of the Invention

The presently claimed and disclosed invention relates, in general, to dual action, single action and soluble heparin/heparosan synthases and, more particularly, to dual action, single action and soluble heparin/heparosan synthases obtained from Pasteurella multocida. The presently claimed and disclosed invention also relates to heparosan, heparin and heparin-like molecules produced according to recombinant techniques and methods of using such molecules. The presently claimed and disclosed invention also relates to methods, and molecules produced according to such methods, for using the presently claimed and disclosed novel synthetic or artificial soluble and/or single action versions of wild type heparosan and/or heparin synthases. The presently claimed and disclosed invention also relates to methods, and molecules produced according to such methods, for using the presently claimed and disclosed heparosan and/or heparin synthases for polymer grafting and the production of non-naturally occurring chimeric polymers incorporating stretches of one or more acidic GAG molecules, such as heparin, chondroitin, hyaluronan, and/or heparosan.

2. Background Information Relating to this Application

Glycosaminoglycans [GAGs] are long linear polysaccharides consisting of disaccharide repeats that contain an amino sugar and are found in most animals. Chondroitin [β(1, 4)GlcUA-β(1, 3)GalNAc]ₙ, heparin/heparosan [β1, 4)GlcUA-[α(1, 4)GlcNAc]ₙ, and hyaluronan [β(1, 4)GlcUA-β(1, 3)GlcNAc]ₙ, are the three most prevalent GAGs found in humans and are also the only known acidic GAGs. Chondroitin and heparin typically have n=20 to 100, while hyaluronan typically has n=10³. Chondroitin and heparin are synthesized as glycoproteins and are sulfated at various positions in vertebrates. Hyaluronan is not sulfated in vertebrates. A substantial fraction of the GlcUA residues of heparin and chondroitin are epimerized to form iduronic acid. A simplified nomenclature has been developed for these GAGs. For example, heparin/heparosan's structure is noted as β4-GlcUA-α4-GlcNAc.

The capsular polysaccharide produced by the Type D strain of Pasteurella multocida is N-acetyl heparosan (heparosan is unmodified heparin—i.e. sulfation or epimerization have not occurred). In vertebrates, one or more modifications including O-sulfation of certain hydroxyls, deacetylation and subsequent N-sulfation, or epimerization of glucuronic acid to iduronic acid modifies the precursor N-acetyl heparosan to heparin/heparan. Hereinafter, for convenience and/or ease of discussion, heparin and/or heparosan are defined as polymers having the β4GlcUA-α4GlcNAc backbone.

With respect to related microbial heparin/heparosan synthases, the E. coli K5 heparin glycosyltransferases, KfiA (SEQ ID NO:7) and KfiC (SEQ ID NO:8), have been identified by genetic and biochemical means. These K5 glycosyltransferases synthesize heparosan (unsulfated and unepimerased heparin) in vivo. The KfiA and KfiC require KfiB (SEQ ID NO:9), an accessory protein, with unknown function in order to synthesize heparosan, however. In vitro, the reactions are limited to adding one or two sugars; as such, it appears that some co-factor or reaction condition is missing—thus, extended polymerization does not occur in vitro when KfiA, KfiB, and KfiC are used. As such, the presently claimed and disclosed heparosan/heparin synthases provide a novel heretofore unavailable means for recombinantly producing heparin (the sulfated and epimerized molecule). In contrast to the presently disclosed and claimed heparin synthases, it appears that K5 requires two proteins, KfiA and KfiC, to transfer the sugars of the disaccharide repeat to the growing polymer chain. The presently claimed and disclosed heparin synthases (designated “pmHS1 and pmHS2”), in one embodiment, are dual action enzymes capable of transferring both sugars of the growing heparin polymer chain. These enzymes polymerize heparosan in vivo and in vitro.

Heparin acts as an anticoagulant and is used to avoid coagulation problems during extra corporal circulation and surgery as well as for treatment after thrombosis has been diagnosed. Heparin is used in the prevention and/or treatment of deep venous thrombosis, pulmonary embolism, mural thrombus after myocardial infarction, post thrombolytic coronary rethrombosis, unstable angina, and acute myocardial infarction. In addition to its use as a treatment for various medical conditions, heparin is also used to coat medical instruments and implants, such as stents, to prevent blood clotting. Using heparin to coat various medical items eliminates the need to prescribe anti-clotting medication.

Where heparin is used to treat medical conditions, such as those described above, two different methods and two different types of heparin are used. Methods include (1) intravenous infusion of standard heparin, and (2) injection of low molecular mass heparin. Patients undergoing intravenous infusion are hospitalized and the activated partial thromboplastin time (aPTT) is monitored. Intravenous infusion requires that the patient remain hospitalized until warfarin is administered to achieve an International Normalized Ratio (INR) between 2.0 and 3.0 often resulting in a three to seven day hospital stay. The alternative treatment involves twice daily injections of low-molecular-weight heparin. The injection treatment allows the patient to self-administer or have a visiting nurse or family member administer the injections.

Low molecular weight heparin has a molecular weight of 1,000 to 10,000 Daltons as compared to the molecular weight of standard heparin of 5,000 to 30,000 Daltons. Low molecular weight heparin binds less strongly to protein than standard heparin, has enhanced bioavailability, interacts less with platelets and yields more predictable blood levels. The predictability of blood levels eliminates the need to monitor the aPPT. In addition, low molecular weight heparin offers a lower likelihood of bleeding and no reports of thrombocytopenia or osteoporosis have been issued with respect to low molecular weight heparin.

In the presently claimed and disclosed invention, pmHS1 and pmHS2 (approximately 70% identical at the amino acid level) are identified (P. multocida Heparin Synthase). pmHS1 and pmHS2 are the first dual action microbial heparin synthases to be identified and molecularly cloned from any source. These enzymes are also shown herein to have particular utility and use, in one embodiment, as catalysts for the formation of heparin and “heparin-like” molecules. With respect to the pmHS1 and pmHS2 enzymes, a single polypeptide is responsible for the copolymerization of the GlcUA and GlcNAc sugars—i.e. the enzymes are dual action enzymes as opposed to the single action nature of the three enzymes of the E. coli K5 heparosan biosynthesis locus (KfiA, KfiC, KfiB) that are required for heparin production. Hereinafter, improved recombinant soluble versions and single action catalysts of the pmHS1 and pmHS2 enzymes are also disclosed.

### SUMMARY OF THE INVENTION

The presently claimed and disclosed invention relates, in general, to dual and single action heparin synthases and, more particularly, to dual and single action heparin synthases obtained from Pasteurella multocida. The presently claimed and disclosed invention also relates to improved soluble versions of the above catalysts. The presently claimed and disclosed invention also relates to heparosan, heparin and heparin-like molecules produced according to recombinant techniques and methods of using such molecules. The presently claimed and disclosed invention also relates to methods, and molecules produced according to such methods, for using the presently claimed and disclosed heparosan and/or heparin synthases for polymer grafting and the production of non-naturally occurring chimeric polymers incorporating stretches of one or more acidic GAG molecules, such as heparin, chondroitin, hyaluronan, and/or heparosan.

It is an object of the presently claimed and disclosed invention to provide a purified nucleic acid segment comprising at least one of: (a) a coding region encoding enzymatically active, soluble heparin synthase; (b) a purified nucleic acid segment encoding an enzymatically active, soluble heparin synthase isolated from Pasteurella multocida; (c) a purified nucleic acid segment encoding the soluble heparin synthase of SEQ ID NO:13 or 15; (d) a purified nucleic acid segment encoding an enzymatically active, soluble heparin synthase, wherein the enzymatically active, soluble heparin synthase is at least 70% identical to SEQ ID NO:13 or 15; (e) a purified nucleic acid segment comprising a nucleotide sequence in accordance with SEQ ID NO:12 or 14; (f) a purified nucleic acid segment capable of hybridizing to the nucleotide sequence of SEQ ID NO:12 or 14 under low, medium or high stringency conditions; (g) a purified nucleic acid segment having semiconservative or conservative amino acid changes or being a truncated segment when compared to the nucleotide sequence of SEQ ID NO:12 or 14; (h) a purified nucleic acid segment having at least one nucleic acid segment sufficiently duplicative of the nucleic acid segment in accordance with SEQ ID NO:12 or 14 to allow possession of the biological property of encoding for a soluble Pasteurella multocida heparin synthase; (i) a purified nucleic acid segment encoding an enzymatically active, soluble heparin synthase, wherein the enzymatically active, soluble heparin synthase is a fragment of SEQ ID NO:2, 4, 6, 13, 15 or 34; and (j) a purified nucleic acid segment comprising a fragment of a nucleic acid sequence in accordance with SEQ ID NO:1, 3, 5, 12, 14 or 33, and wherein the purified nucleic acid segment encodes an enzymatically active, soluble heparin synthase. The purified nucleic acid segment may be provided in a recombinant vector selected from the group consisting of a plasmid, cosmid, phage, integrated cassette or virus vector. The recombinant vector containing the purified nucleic acid segment is used to electroporate, transform or transduce a host cell to produce a recombinant host cell having the recombinant vector. Preferably, the recombinant host cell produces heparin or heparin synthase, and the heparin polymer may have a modified structure or modified size distribution. In addition, the recombinant host cell may further comprise at least one of an epimerase, a sulfotransferase, and combinations thereof.

It is another object of the presently claimed and disclosed invention, while achieving the before-stated object, to provide a method for producing a heparin polymer in vitro. The method includes providing a soluble heparin synthase and placing the soluble heparin synthase in a reaction mixture containing UDP-GlcNAc and UDP-GlcUA and at least one divalent metal ion suitable for the synthesis of a heparin polymer, followed by extracting the heparin polymer out of the reaction mixture. Preferably, the soluble heparin synthase is encoded by the purified nucleic acid segment described in the paragraph above.

It is another object of the presently disclosed and claimed invention, while achieving the before-stated objects, to provide a purified nucleic acid segment comprising at least one of: (a) a coding region encoding a modified heparin synthase, wherein the modified heparin synthase is capable of adding at least one of GlcUA and GlcNAc to a heparin polymer; (b) a coding region encoding a modified soluble heparin synthase, wherein the modified soluble heparin synthase is capable of adding at least one of GlcUA and GlcNAc to a heparin polymer; (c) a purified nucleic acid segment encoding a modified heparin synthase of SEQ ID NO:25 or 27 wherein the modified heparin synthase is capable of adding at least one of GlcUA and GlcNAc to a heparin polymer; (d) a purified nucleic acid segment encoding a modified heparin synthase having at least about 70% identity to SEQ ID NO:25 or 27 and wherein the modified heparin synthase is capable of adding at least one of GlcUA and GlcNAc to a heparin polymer; (e) a purified nucleic acid segment comprising a nucleotide sequence in accordance with SEQ ID NO:24 or 26; (f) a purified nucleic acid segment capable of hybridizing to the nucleotide sequence of SEQ ID NO:24 or 26 under low, medium or high stringency conditions; (g) a purified nucleic acid segment having semiconservative or conservative amino acid changes or being a truncated segment when compared to the nucleotide sequence of SEQ ID NO:24 or 26; (h) a purified nucleic acid segment having at least one nucleic acid segment sufficiently duplicative of the nucleic acid segment in accordance with SEQ ID NO:24 or 26 to allow possession of the biological property of encoding for a single-action of Pasteurella multocida heparin synthase; (i) a purified nucleic acid segment encoding a modified heparin synthase, wherein the modified heparin synthase is capable of adding at least one of GlcUA and GlcNAc to a heparin polymer, and wherein the modified heparin synthase is at least about 70% identical to SEQ ID NO:2, 4, 6, 13, 15 or 34; and (j) a purified nucleic acid segment comprising a nucleic acid sequence at least about 70% identical to SEQ ID NO:1, 3, 5, 12, 14 or 33, and wherein the purified nucleic acid segment encodes a modified heparin synthase capable of adding at least one of GlcUA and GlcNAc to a heparin polymer. The purified nucleic acid segment may be provided in a recombinant vector selected from the group consisting of a plasmid, cosmid, phage, integrated cassette or virus vector. The recombinant vector containing the purified nucleic acid segment is used to electroporate, transform or transduce a host cell to produce a recombinant host cell having the recombinant vector. Preferably, the recombinant host cell produces heparin, and the heparin polymer may have a modified structure or modified size distribution. In addition, the recombinant host cell may produce a modified heparin synthase capable of adding at least one of GlcUA and GlcNAc to a heparin polymer.

It is another object of the presently claimed and disclosed invention, while achieving the before-stated object, to provide a method for enzymatically producing a polymer. The method includes providing a functional acceptor, wherein the functional acceptor has at least two sugar units selected from the group consisting of uronic acid and hexosamine. The method also includes providing a modified heparin/heparosan synthase capable of elongating the functional acceptor, wherein the modified heparin/heparosan synthase is a single action glycosyltransferase capable of adding only one of GlcUA or GlcNAc and has an amino acid sequence encoded by the nucleic acid segment of described in the paragraph above. The method further includes providing at least one of UDP-GlcUA, UDP-GlcNAc and UDP-sugar analogs such that the modified heparin/heparosan synthase elongates the functional acceptor in a single step manner so as to provide a polymer.

In the method, uronic acid is further defined as a uronic acid selected from the group consisting of GlcUA, IdoUA, and GalUA, and hexosamine is further defined as a hexosamine selected from the group consisting of GlcNAc, GalNAc, GlcN and GalN. The functional acceptor may have about three or four sugar units.

Other objects, features and advantages of the present invention will become apparent from the following detailed description when read in conjunction with the accompanying drawings and appended claims.

### BRIEF DESCRIPTION OF THE SEVERAL VIEWS OF THE DRAWINGS

FIG. 1 graphically depicts Sequence Similarity of pmHS1 with KfiA and KfiC. Elements of the Pasteurella pmHS1 heparosan synthase, HSA (containing residues 91-240; (SEQ ID NO:10)) and HSA (containing residues 441-540; (SQE ID NO:11)) are very similar to portions of two proteins from the E. coli K5 capsular locus (residues 75-172 of KfiA; residues 262-410 of KfiC) as shown by this modified Mutalin alignment (ref. 21; numbering scheme corresponds to the pmHS1 sequence). The HSA and HSB elements may be important for hexosamine transferase or for glucuronic acid transferase activities, respectively. (con, consensus symbols: asterisks, [K or R] and [S or T]; %, any one of F,Y,W; $, any one of L,M; !, any one of I,V; #, any one of E,D,Q,N).

FIG. 2 depicts pmHS1 Activity Dependence on Acceptor and Enzyme Concentration. Various amounts of crude membranes containing the full-length enzyme, pmHS1¹⁻⁶¹⁷, were incubated in 50 μl of buffer containing 50 mM Tris, pH 7.2, 10 mM MgCl₂, 10 mM MnCl₂, 500 μM UDP-[¹⁴C]GlcUA (0.15 μCi), and 500 μM UDP-GlcNAc. Three parallel sets of reactions were performed with either no acceptor (circles) or two concentrations of heparosan polymer acceptor (uronic acid: 0.6 μg, squares; 1.7 μg, triangles). After 40 min, the reaction was terminated and analyzed by paper chromatography. The background incorporation due to vector membranes alone (630 μg total protein; not plotted) with the high concentration of acceptor was 75 dpm [¹⁴C]GlcUA. The activity of pmHS1 is greatly stimulated by exogenous acceptor.

FIG. 3 graphically depicts Sequence Similarity of pmHS1 and pmHS2. The two distinct Pasteurella heparosan synthases, pmHS1 and pmHS2 are very similar (approximately 73% identical) as shown by this differential Multalin alignment; numbering scheme corresponds to the pmHS2 sequence). (con, consensus symbols: asterisks, [K or R] and [S or T]; %, any one of F,Y,W; $, any one of L,M; !, any one of I,V; #, any one of E,D,Q,N).

FIG. 4 graphically depicts Western Blot Analysis of pmHS1 and pmHS2. Membrane preparations from recombinant E. coli with plasmids containing pmHS1, pmHS2 or no insert (vec) were analyzed with an anti-peptide antibody. The relevant region spanning the 95.5 to 55 kDa standards is shown. As predicted from the deduced sequence, the larger pmHS2 polypeptide migrates slower than pmHS1.

FIG. 5 graphically depicts Acceptor Sugar Usage by Recombinant pmHS2. Increasing amounts of the recombinant pmHS2 protein in membrane preparations were assayed in the presence (▪) or absence (▴) of acceptor polymer. The acceptor increases the incorporation rate by about 2.5-fold. No significant incorporation is observed with the vector membranes. In contrast, pmHS1 is stimulated by ˜7- to 25-fold by exogenous acceptor.

FIG. 6 depicts Gel Filtration Analysis of pmHS1 and pmHS2 Products. The crude membranes containing pmHS2 (A) and pmHS1 (B) (0.36 mg total protein) were incubated with 500 μM UDP-[¹⁴C]GlcUA (0.15 μCi) and 500 μM UDP-GlcNAc in a 75 μl reaction volume either in the presence (thick line) or absence (thin line) of Type D acceptor polymer (0.4 μg uronic acid; 12.8 min elution time). After 60 minutes the samples were analyzed on the PolySep 4000 column (DPS, disintegrations per second; calibration elution times in minutes: void volume, 9.8; 580 kDa dextran, 12.3; 145 kDa dextran, 12.75, totally included volume, 16.7). The background signal for experiments using vector control membranes (not shown) was<6 DPS throughout the relevant polymer region. The activity of pmHS2 is not greatly stimulated by exogenous acceptor and the final polymer size distribution is lower molecular weight than observed for pmHS1.

FIG. 7 depicts Southern. Blot Analysis of pmHS1 and pmHS2 in chromosome of various Pasteurella multocida isolates. Duplicate blots of digested genomic DNA (either HinDIII or NcoI/XhoI, as noted in figure) from a Type A strain (A) and two Type D strains (D, D′) were hybridized with either the pmHS1 or the pmHS2 probe. The relevant portions of the blots are shown. The Type D strains, but not Type A, possess a pmHS1 gene, while all three strains have a pmHS2 gene. The pmHS2 resides in the same chromosomal location in all three strains.

FIG. 8 graphically depicts a schematic model of the known Pasteurella Glycosaminoglycan synthases. The native enzymes contain two different sugar transferase sites (−Tase) and a membrane association region (mem). The pmHAS and pmCS are similarly organized and have 90% identical sequence. On the other hand, pmHS1 and pmHS2 do not have the same structure or sequence similarity to pmHAS and pmCS. Removal of a “mem” section creates a soluble enzyme.

FIG. 9 graphically depicts a schematic model of a general method to convert a heparosan synthase (dual-action polymerizing activity) to single-action mutants. Mutation of critical residue(s) in one domain will inactivate that domain, but the other domain remains unaffected. Thus, useful single sugar transfer reactions are then possible.

### DETAILED DESCRIPTION OF THE INVENTION

Before explaining at least one embodiment of the invention in detail, it is to be understood that the invention is not limited in its application to the details of construction and the arrangement of the components set forth in the following description, claims, examples, or illustrated in the drawings. The invention is capable of other embodiments or of being practiced or carried out in various ways. Also, it is to be understood that the phraseology and terminology employed herein is for purpose of description and should not be regarded as limiting.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not necessarily mean that the material is 100% purified so as to exclude any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a Heparin or Heparosan Synthase (“HS”) coding sequence yet is isolated away from, or purified free from, unrelated genomic DNA, for example, total Pasteurella multocida or, for example, mammalian host genomic DNA. Included within the term “DNA segment” are DNA segments and smaller fragments of such segments, and also recombinant vectors including, for example, plasmids, cosmids, phage, viruses, and the like.

Similarly, a DNA segment comprising an isolated or purified pmHS1 (Pasteurella multocida Heparin Synthase) gene or a pmHS2 gene refers to a DNA segment including HS coding sequences isolated substantially away from other naturally occurring genes or protein encoding sequences. In this respect, the term “gene” is used for simplicity to refer to a functional protein, polypeptide or peptide encoding unit. As will be understood by those in the art, this functional term includes genomic sequences, cDNA sequences or combinations thereof. “Isolated substantially away from other coding sequences” means that the gene of interest, in this case pmHS1 or pmHS2, forms the significant part of the coding region of the DNA segment, and that the DNA segment does not contain large portions of naturally-occurring coding DNA, such as large chromosomal fragments or other functional genes or DNA coding regions. Of course, this refers to the DNA segment as originally isolated, and does not exclude genes or coding regions later added to, or intentionally left in the segment by the hand of man.

Due to certain advantages associated with the use of prokaryotic sources, one will likely realize the most advantages upon isolation of the HS genes from Pasteurella multocida. One such advantage is that, typically, eukaryotic enzymes may require significant post-translational modifications that can only be achieved in an eukaryotic host. This will tend to limit the applicability of any eukaryotic HS genes that are obtained. Additionally, such eukaryotic HS genes are dainty, fragile, and difficult, if not impossible, to transfer into prokaryotic hosts for large scale polymer production. Moreover, those of ordinary skill in the art will likely realize additional advantages in terms of time and ease of genetic manipulation where a prokaryotic enzyme gene is sought to be employed. These additional advantages include (a) the ease of isolation of a prokaryotic gene because of the relatively small size of the genome and, therefore, the reduced amount of screening of the corresponding genomic library; and (b) the ease of manipulation because the overall size of the coding region of a prokaryotic gene is significantly smaller due to the absence of introns. Furthermore, if the product of the HS genes (i.e., the enzyme) requires posttranslational modifications or cofactors, these would best be achieved in a similar prokaryotic cellular environment (host) from which the gene was derived.

Preferably, DNA sequences in accordance with the present invention will further include genetic control regions which allow the expression of the sequence in a selected recombinant host. Of course, the nature of the control region employed will generally vary depending on the particular use (e.g., cloning host) envisioned.

In particular embodiments, the invention concerns isolated DNA segments and recombinant vectors incorporating DNA sequences which encode a HS gene such as pmHS1 or pmHS2, or active fragment thereof. In the case of pmHS1, the isolated DNA segments and recombinant vectors incorporating DNA sequences which include within their amino acid sequences an amino acid sequence in accordance with SEQ ID NO:2 or SEQ ID NO:4; for pmHS2, an amino acid sequence in accordance with SEQ ID NO: 6 or SEQ ID NO:34. In the case of soluble pmHS1, an amino acid sequence in accordance with SEQ ID NO:13 or SEQ ID NO:15; in the case of single-action mutants of pmHS1, an amino acid sequence in accordance with SEQ ID NO:25 or SEQ ID NO:27. Moreover, in other particular embodiments, the invention concerns isolated DNA segments and recombinant vectors incorporating DNA sequences which encode a gene that includes within its amino acid sequence the amino acid sequence of an HS gene or DNA, and in particular to a HS gene or cDNA, corresponding to Pasteurella multocida Heparin Synthases—pmHS1 and pmHS2. For example, where the DNA segment or vector encodes a full length HS protein, or is intended for use in expressing the HS protein, preferred sequences are those which are essentially as set forth in SEQ ID NO:2 or SEQ ID NO:4 or SEQ ID NO:6 or SEQ ID NO:34. Additionally, sequences that have at least one or more amino acid motifs (discussed in detail hereinafter) and encode a functionally active heparosan/heparin synthase are contemplated for use.

The presently claimed and disclosed pmHS1 includes SEQ ID NOS:1 and 3 (nucleotide sequence) and SEQ ID NOS:2 and 4 (amino acid sequences) that have been assigned GenBank Accession Nos. AF425591 and AF438904, respectively. The presently claimed and disclosed pmHS2 includes SEQ ID NOS:5 and 33 (nucleotide sequences) and SEQ ID NOS:6 and 34 (amino acid sequences) that have been assigned GenBank Accession Nos. AY292199 and AY292200, respectively, for pmHS2 isolated from Type A and Type D, P. multocida, respectively. The presently claimed and disclosed soluble pmHS1 includes SEQ ID NOS:12 and 14 (nucleotide sequence) and SEQ ID NOS: 13 and 15 (amino acid sequence). The presently claimed and disclosed single-action pmHS1 mutants include SEQ ID NOS:24 and 26 (nucleotide sequence) and SEQ ID NOS:25 and 27 (amino acid sequence). Amino acid motifs for enzymatically active heparosan/heparin synthases are disclosed in detail hereinafter.

Nucleic acid segments having heparin synthase activity may be isolated by the methods described herein. The term “a sequence essentially as set forth in SEQ ID NO:2 or 4 or 6 or 13 or 15 or 25 or 27 or 34” means that the sequence substantially corresponds to a portion of SEQ ID NO:2 or 4 or 6 or 13 or 15 or 25 or 27 or 34 and has relatively few amino acids which are not identical to, or a biologically functional equivalent of, the amino acids of SEQ ID NO:2 or 4 or 6 or 13 or 15 or 25 or 27 or 34. The term “biologically functional equivalent” is well understood to those of skill in the art and is embodied in the knowledge that modifications and changes may be made in the structure of a protein or peptide and still obtain a molecule having like or otherwise desirable characteristics. However, it is also well understood by skilled artisans that, inherent in the definition of a biologically functional equivalent protein or peptide, is the concept that there is a limit to the number of changes that may be made within a defined portion of the molecule and still result in a molecule with an acceptable level of equivalent biological activity, and that key active site or structurally vital residues cannot be exchanged (see for example, U.S. Pat. No. 6,355,619, issued to Miller et al. on Mar. 12, 2002, the contents of which are hereby expressly incorporated herein by reference). The term “biologically functional equivalent” is further defined in detail herein as a gene having a sequence essentially as set forth in SEQ ID NO:2 or 4 or 6 or 13 or 15 or 25 or 27 or 34, and that is associated with the ability of prokaryotes to produce heparin/heparosan or a “heparin like” polymer or a heparin synthase polypeptide. For example, pmHS2 is approximately 70% identical to pmHS1 and pmHS2 is shown, hereinafter, to be an enzymatically active heparin/heparosan synthase.

One of ordinary skill in the art would appreciate that a nucleic acid segment encoding enzymatically active heparin synthase may contain conserved or semi-conserved substitutions to the sequences set forth in SEQ ID NOS: 1, 2, 3, 4, 5, 6, 12, 13, 14, 15, 24, 25, 26, 27, 33 or 34 and yet still be within the scope of the invention.

In particular, the art is replete with examples of practitioner's ability to make structural changes to a nucleic acid segment (i.e. encoding conserved or semi-conserved amino acid substitutions) and still preserve its enzymatic or functional activity. See for example: (1) Risler et al. “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (I) Ile and Val; (ii) Leu and Met, (iii) Lys, Arg, and Gln, and (iv) Tyr and Phe.”]; (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . ” Compatible changes can be made]. Each of these articles, to the extent that they provide additional details to one of ordinary skill in the art in the methods of making such conserved or semi-conserved amino acid substitutions, are hereby expressly incorporated herein in their entirety as though set forth herein.

These references and countless others available to one of ordinary-skill in the art, indicate that given a nucleic acid sequence, one of ordinary skill in the art could make substitutions and changes to the nucleic acid sequence without changing its functionality. Also, a substituted nucleic acid segment may be highly identical and retain its enzymatic activity with regard to its unadulterated parent, and yet still fail to hybridize thereto (i.e. spHAS and seHAS, 70% identical yet do not hybridize under standard hybridization conditions as defined hereinafter). Therefore, the ability of two sequences to hybridize to one another can be a starting point for comparison but should not be the only ending point—rather, one of ordinary skill in the art must look to the conserved and semi-conserved amino acid stretches between the sequences between the sequences and also must assess functionality. Thus, given that two sequences may have conserved and/or semi-conserved amino acid stretches, functionality must be assessed.

One of ordinary skill in the art would also appreciate that substitutions can be made to the pmHS1 nucleic acid segment listed in SEQ ID NO: 1 or 3 or 5 or 12 or 14 or 24 or 26 or 33 that do not affect the amino acid sequences they encode or result in conservative or semi-conservative substitutions in the amino acid sequences they encode without deviating outside the scope and claims of the present invention. Standardized and accepted functionally equivalent amino acid substitutions are presented in Table I.

TABLE I

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, | Serine, Threonine, Cysteine, Asparagine, |
| R Groups | Glutamine |
| Negatively Charged R Groups | Aspartic Acid, Glutamic Acid |
| Positively Charged R Groups | Lysine, Arginine, Histidine |

A particular example of conservative or semi-conservative amino acid substitutions resulting in biologically functional equivalents would be SEQ ID NO NOS: 2 and 4, both of which encode a functionally active HS and yet have a single substitution at position 455 (Threonine for Isoleucine), and yet both enzymes are still capable of producing heparosan. Such a conservative or semi-conservative scheme is even more evident when comparing pmHS1 with pmHS2 —they are only ˜70% identical and yet still both produce functionally active HS enzymes.

Another preferred embodiment of the present invention is a purified nucleic acid segment that encodes a protein in accordance with SEQ ID NO:2 or 4 or 6 or 13 or 15 or 25 or 27 or 34 further defined as a recombinant vector. As used herein, the term “recombinant vector” refers to a vector that has been modified to contain a nucleic acid segment (or nucleic acid segments such as more than one copy of SEQ ID NO:2 or 4 or 6 or 13 or 15 or 25 or 27 or 34) that encodes a HS protein, or fragment thereof. The recombinant vector may be further defined as an expression vector comprising one or more promoters operatively linked to said HS encoding nucleic acid segment.

A further preferred embodiment of the present invention is a host cell, made recombinant with a recombinant vector comprising a HS gene. The preferred recombinant host cell may be a prokaryotic cell. In another embodiment, the recombinant host cell is an eukaryotic cell. As used herein, the term “engineered” or “recombinant” cell is intended to refer to a cell into which a recombinant gene, such as a gene encoding HS, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, one or more copies of a genomic gene, or will include genes positioned adjacent to a promoter not naturally associated with the particular introduced gene.

Where one desires to use a host other than Pasteurella, as may be used to produce recombinant heparin/heparosan synthase, it may be advantageous to employ a prokaryotic system such as E. coli, B. subtilis, Lactococcus sp., (see, for example, U.S. patent application Ser. No. 09/469,200, which discloses the production of HA through the introduction of a HAS gene into Bacillus host—the contents of which are expressly incorporated herein in their entirety), or even eukaryotic systems such as yeast or Chinese hamster ovary, African green monkey kidney cells, VERO cells, or the like. Preferably, the host cell will be selected from the group consisting of a Bacillus host such as Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, Bacillus thuringiensis; a Streptomyces host such as Streptomyces lividans or Streptomyces murinus; a gram negative bacteria such as E. coli or Pseudomonas; a fungus or yeast host such as Candida, Kluyveromyces, Pichia, Saccharomyces, Schizosaccharomyces, Yarrowia, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma. The host cell may also be selected from the group consisting of Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, and Trichoderma viride. Of course, where this is undertaken it will generally be desirable to bring the heparin/heparosan synthase gene under the control of sequences which are functional in the selected alternative host. The appropriate DNA control sequences, as well as their construction and use, are generally well known in the art as discussed in more detail hereinbelow.

In preferred embodiments, the heparin/heparosan synthase-encoding DNA segments further include DNA sequences, known in the art functionally as origins of replication or “replicons”, which allow replication of contiguous sequences by the particular host. Such origins allow the preparation of extrachromosomally localized and replicating chimeric segments or plasmids, to which HS DNA sequences are ligated. In more preferred instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

Thus, it will be appreciated by those of ordinary skill in the art that other means may be used to obtain the HS gene or cDNA, in light of the present disclosure. For example, polymerase chain reaction or RT-PCR produced DNA fragments may be obtained which contain full complements of genes or cDNAs from a number of sources, including other strains of Pasteurella or from eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HS, and in a more preferred embodiment, the isolated nucleic acids should encode an amino acid sequence that contains at least one of the HS amino acid motifs described in detail hereinafter.

Once the DNA has been isolated it is ligated together with a selected vector. Virtually any cloning vector can be employed to realize advantages in accordance with the invention. Typical useful vectors include plasmids and phages for use in prokaryotic organisms and even viral vectors for use in eukaryotic organisms. Generally Regarded As Safe (GRAS) organisms are advantageous in that one can augment the strain's ability to synthesize heparin/heparosan through gene dosaging (i.e., providing extra copies of the Heparosan synthase gene by amplification) and/or the inclusion of additional genes to increase the availability of the heparin/heparosan precursors UDP-GlcUA and UDP-GlcNAc and/or the inclusion of genes that include enzymes that will make modifications (such as sulfation and epimerization) to the heparosan polymer in order to convert it to heparin. Sugar precursors are made by the enzymes with UDP-glucose dehydrogenase and UDP-N-acetylglucosamine pyrophosphorylase activity, respectively. The inherent ability of a bacterium to synthesize heparin/heparosan is also augmented through the formation of extra copies, or amplification, of the plasmid that carries the heparin/heparosan synthase gene. This amplification can account for up to a 10-fold increase in plasmid copy number and, therefore, the HS gene copy number.

Another procedure that would further augment HS gene copy number is the insertion of multiple copies of the gene into the plasmid. Another technique would include integrating the HS gene into chromosomal DNA. This extra amplification would be especially feasible, since the HS gene size is small. In some scenarios, the chromosomal DNA-ligated vector is employed to transfect the host that is selected for clonal screening purposes such as E. coli or Bacillus, through the use of a vector that is capable of expressing the inserted DNA in the chosen host. In certain instances, especially to confer stability, genes such as the HS gene, may be integrated into the chromosome in various positions in an operative fashion. Unlike plasmids, integrated genes do not need selection pressure for maintenance of the recombinant gene.

In certain other embodiments, the invention concerns isolated DNA segments and recombinant vectors that include within their sequence a nucleic acid sequence essentially as set forth in SEQ ID NO:1 or 3 or 5 or 12 or 14 or 24 or 26 or 33. The term “essentially as set forth in SEQ ID NO:1 or 3 or 5 or 12 or 14 or 24 or 26 or 33” is used in the same sense as described above with respect to the amino acid sequences and means that the nucleic acid sequence substantially corresponds to a portion of SEQ ID NO:1 or 3 or 5 or 12 or 14 or 24 or 26 or 33, and has relatively few codons that are not identical, or functionally equivalent, to the codons of SEQ ID NO:1 or 3 or 5 or 12 or 14 or 24 or 26 or 33 and encodes a enzymatically active HS or single-action fragment of HS. The term “functionally equivalent codon” is used herein to refer to codons that encode the same amino acid, such as the six codons for arginine or serine, and also refers to codons that encode biologically equivalent amino acids. “Biologically Equivalent Amino Acids” of Table I refers to residues that have similar chemical or physical properties that may be easily interchanged for one another.

It will also be understood that amino acid and nucleic acid sequences may include additional residues, such as additional N- or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and enzymatic activity is concerned. The addition of terminal sequences particularly applies to nucleic acid sequences which may, for example, include various non-coding sequences flanking either of the 5′ or 3′ portions of the coding region or may include various internal sequences, which are known to occur within genes.

Likewise, deletion of certain portions of the polypeptide can be desirable. For example, functional truncated versions of pmHAS, the Pasteurella hyaluronan synthase, missing the carboxyl terminus enhances the utility for in vitro use. The truncated pmHAS enzyme is a soluble protein that is easy to purify in contrast to the full-length protein (972 residues). Also, the expression level of the enzyme increases greatly as the membrane is not overloaded. It was previously predicted that a truncated version of pmHS1 would also be useful (see U.S. Ser. No. 10/142,143, which has previously been incorporated herein by reference). Such a truncated version would also be highly soluble and increase expression of the enzyme; the native membrane proteins are found in low levels and are not soluble without special treatment with detergents. A truncated, soluble version of pmHS1 (pmHS1^{K45M-617} or pmHS1^{I77M-617} (SEQ ID NOS:13 and 15, respectively)) shown and described herein and falls within the scope of the presently claimed and disclosed invention. However, the truncation to form a soluble pmHS1 described herein is a different truncation than that predicted from the truncated, soluble forms of pmHAS; the truncations that produce pmHAS¹⁻⁹⁷² to pmHAS¹⁻⁷⁰³ are carboxyl-terminal deletions versus the amino-terminal deletion that produces pmHS1^{K45M-617} and pmHS1^{I77M-617}.

Allowing for the degeneracy of the genetic code as well as conserved and semi-conserved substitutions, sequences which have between about 40% and about 80%; or more preferably, between about 80% and about 90%; or even more preferably, between about 90% and about ⁹⁹% of nucleotides which are identical to the nucleotides of SEQ ID NO:1 or 3 or 5 or 12 or 14 or 24 or 26 or 33 will be sequences which are “essentially as set forth in SEQ ID NO:1 or 3 or 5 or 12 or 14 or 24 or 26 or 33”. In one embodiment, the sequences will be 40%-42% identical, 42%-44% identical, 44%-46% identical, 46%-48% identical, 48%-50% identical, 50%-52% identical, 52%-54% identical, 54%-56% identical, 56%-58% identical, 58%-60% identical, 60%-62% identical, 62%-64% identical, 64%-66% identical, 66%-68% identical, 68%-70% identical, 70%-72% identical, 72%-74% identical, 74%-76% identical, 76%-78% identical, 78%-80% identical, 80%-82% identical, 82%-84% identical, 84%-86% identical, 86%-88% identical, 88%-90% identical, 90%-92% identical, 92%-94% identical, 94%-96% identical, 96%-98% identical, or 98%-100% identical to SEQ ID NO:1 or 3 or 5 or 12 or 14 or 24 or 26 or 33. In a preferred embodiment, the sequences would be either 40% or 70% identical. Sequences which are essentially the same as those set forth in SEQ ID NO:1 or 3 or 5 or 12 or 14 or 24 or 26 or 33 may also be functionally defined as sequences which are capable of hybridizing to a nucleic acid segment containing the complement of SEQ ID NO:1 or 3 or 5 or 12 or 14 or 24 or 26 or 33 under standard or less stringent hybridizing conditions. Suitable standard hybridization conditions will be well known to those of skill in the art and are clearly set forth hereinbelow. As certain domains and active sites are formed from a relatively small portion of the total polypeptide, these regions of sequence identity or similarity may be present only in portions of the gene. Additionally, sequences which are “essentially as set forth in SEQ ID NO:1 or 3 or 5 or 12 or 14 or 24 or 26 or 33” will include those amino acid sequences that have at least one of the heparin enzyme amino acid motifs (described hereinafter in detail) and that also retain the functionality of an enzymatically active HS or single-action fragment thereof.

The polypeptides of the present invention have at least 20%, preferably at least 25%, more preferably at least 30%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 90%, and most preferably at least 100% of the single- or dual-action HS activity of the mature polypeptide of SEQ ID NO:2.

As is well known to those of ordinary skill in the art, most of the amino acids in a protein are present to form the “scaffolding” or general environment of the protein. The actual working parts responsible for the specific desired catalysis are usually a series of small domains or motifs. Thus, a pair of enzymes that possess the same or similar motifs would be expected to possess the same or similar catalytic activity, thus they are functionally equivalent. Utility for this hypothetical pair of enzymes may be considered interchangeable unless one member of the pair has a subset of distinct, useful properties. Similarly, certain non-critical motifs or domains may be dissected from the original, naturally occurring protein and function will not be affected; removal of non-critical residues does not perturb the important action of the remaining critical motifs or domains. By analogy, with sufficient planning and knowledge, it is possible to translocate motifs or domains from one enzyme to another polypeptide to confer the new enzyme with desirable characteristics intrinsic to the domain or motif. Such motifs for HS are disclosed in particularly hereinafter.

Similarly, certain critical motifs or domains may be changed (mutated) or dissected from the original, naturally occurring protein to thereby affect function; removal of critical residues will perturb the important action of the remaining critical motifs or domains. Such motifs for HS are disclosed in particularly hereinafter. The pmHS1 and pmHS2 enzymes in the natural state are dual action enzymes with two separate active sites or domains. Theoretically, if the sites are relatively functionally independent, then the alteration of one site or domain will not destroy the activity of the other unmutated site. The theory is held to be true in the presently disclosed and claimed invention. Such is the case with mutated, soluble versions of pmHS1 (such as thioredoxin fusions containing full-length pmHS1-D¹⁸¹N-D¹⁸³N (SEQ ID NO:25) or pmHS1-D⁴⁴⁴N-D⁴⁴⁶N (SEQ ID NO:27)) are shown and described herein and fall within the scope of the presently claimed and disclosed invention of single action transferases.

The term “standard hybridization conditions” as used herein, is used to describe those conditions under which substantially complementary nucleic acid segments will form standard Watson-Crick base-pairing. A number of factors are known that determine the specificity of binding or hybridization, such as pH, temperature, salt concentration, the presence of agents such as formamide and dimethyl sulfoxide, the length of the segments that are hybridizing, and the like. When it is contemplated that shorter nucleic acid segments will be used for hybridization, for example fragments between about 14 and about 100 nucleotides, salt and temperature preferred conditions for overnight standard hybridization will include 1.2-1.8×HPB (High Phosphate Buffer) at 40-50° C. or 5×SSC (Standard Saline Citrate) at 50° C. Washes in low salt (10 mM salt or 0.1×SSC) are used for stringent hybridizations with room temperature incubations of 10-60 minutes. Washes with 0.5×to 1×SSC, 1% Sodium dodecyl sulfate at room temperature are used in lower stringency washes for 15-30 minutes. For all hybridizations: (where 1×HPB=0.5 m NaCl, 0.1 m Na₂HPO₄, 5 mM EDTA, pH 7.0) and (where 20×SSC=3 m NaCl, 0.3 m Sodium Citrate with pH 7.0).

For long probes of at least 100 nucleotides in length, very low to very high stringency conditions are defined as prehybridization and hybridization at 42° C. in 5× SSPE, 0.30% SDS, 200 mg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6× SCC plus 0.1% SDS for 15 minutes and twice each for 15 minutes using 6× SSC at 5° C. to 10° C. below the calculated Tₘ.

Naturally, the present invention also encompasses DNA segments which are complementary, or essentially complementary, to the sequence set forth in SEQ ID NOS:1, 3, 5, 12, 14, 24, or 26 or 33. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment of SEQ ID NO:1, 3, 5, 12, 14, 24, or 26 or 33 under the above-defined standard hybridization conditions.

The nucleic acid segments of the present invention, regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. For example, functional spHAS-(Histidine)₆ and x1HAS1-(Green Fluorescent Protein) fusion proteins have been reported. It is therefore contemplated that a nucleic acid fragment of almost any length may be employed, with the total length preferably being limited by the ease of preparation and use in the intended recombinant DNA protocol.

Naturally, it will also be understood that this invention is not limited to the particular nucleic acid and amino acid sequences of SEQ ID NOS:1, 2, 3, 4, 5, 6, 12, 13, 14, 15, 24, 25, 26,27, 33 or 34. Recombinant vectors and isolated DNA segments may therefore variously include the HS coding regions themselves, coding regions bearing selected alterations or modifications in the basic coding region, or they may encode larger polypeptides which nevertheless include HS coding regions or may encode biologically functional equivalent proteins or peptides which have variant amino acid sequences.

Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

The DNA segments of the present invention encompass biologically functional equivalent HS proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the HS protein or to test HS mutants in order to examine HS activity at the molecular level.

Also, specific changes to the HS coding sequence will result in the production of heparin/heparosan having a modified size distribution or structural configuration. One of ordinary skill in the art would appreciate that the HS coding sequence can be manipulated in a manner to produce an altered HS which in turn is capable of producing heparin/heparosan having differing polymer sizes and/or functional capabilities. The utility of such a modified polymer is easily appreciated from the above “Background of the Invention.” For example, the HS coding sequence may be altered in such a manner that the HS has analtered sugar substrate specificity so that the HS creates a new heparin/heparosan-like chimeric polymer incorporating a different structure via the inclusion of a previously unincorporated sugar or sugar derivative. This newly incorporated sugar results in a modified heparin/heparosan having different and unique functional properties. As will be appreciated by one of ordinary skill in the art given the HS coding sequences, changes and/or substitutions can be made to the HS coding sequence such that these desired properties and/or size modifications can be accomplished.

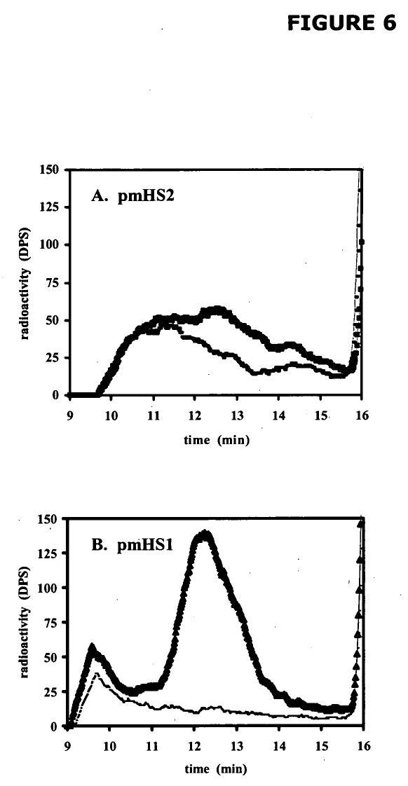

Basic knowledge on the substrate binding sites (e.g. the UDP-GlcUA site or UDP-GlcNAc site or oligosaccharide acceptor site) of pmHS1 or pmHS2 allows the targeting of residues for mutation to change the catalytic properties of the site. The identity of important catalytic residues of pmHAS, another GAG synthase, have recently been elucidated (Jing & DeAngelis, 2000, the contents of which are expressly incorporated herein in their entirety). Appropriate changes at or near these residues alters UDP-sugar binding. Changes of residues in close proximity should allow other precursors to bind instead of the authentic heparin/heparosan sugar precursors; thus a new, modified polymer is synthesized. Polymer size changes are caused by differences in the synthase's catalytic efficiency or changes in the acceptor site affinity. Polymer size changes have been made in seHAS and spHAS (U.S. patent application Ser. Nos. 09/559,793 and 09/469,200, the contents of which are expressly incorporated herein by reference) as well as the vertebrate HAS, xlHAS1 (DG42) (Pummill & DeAngelis, 2003, the contents of which are expressly incorporated herein in their entirety) by mutating various residues. As pmHS1 is a more malleable, robust enzyme than these other enzymes, similar or superior versions of mutant pmHS1 or pmHS2 which synthesize modified polymers are easily produced. In addition, the different sequence heparosan synthases can be used to make different sized polymers. That is, pmHS1 produces a larger polymer than pmHS2 (see FIG. 6).

The term “modified structure” as used herein denotes a heparin/heparosan polymer containing a sugar or derivative not normally found in the naturally occurring heparin/heparosan polypeptide. The term “modified size distribution” refers to the synthesis of heparin/heparosan molecules of a size distribution not normally found with the native enzyme; the engineered size could be much smaller or larger than normal.

One of ordinary skill in the art given this disclosure would appreciate that there are several ways in which the size distribution of the heparin/heparosan polymer made by the HS could be regulated to give different sizes. First, the kinetic control of product size can be altered by environmental factors such as decreasing temperature, decreasing time of enzyme action and/or by decreasing the concentration of one or both sugar nucleotide substrates. Decreasing any or all of these variables will give lower amounts and smaller sizes of heparin/heparosan product. The disadvantages of these extrinsic approaches are that the yield of product is also decreased and it is difficult to achieve reproducibility from day to day or batch to batch. Secondly, the alteration of the intrinsic ability of the enzyme to synthesize a large or small heparin/heparosan product. Changes to the protein are engineered by recombinant DNA technology, including substitution, deletion and addition of specific amino acids (or even the introduction of prosthetic groups through metabolic processing). Such changes that result in an intrinsically slower enzyme then allow for more reproducible control of heparin/heparosan size by kinetic means. The final heparin/heparosan size distribution is determined by certain characteristics of the enzyme that rely on particular amino acids in the sequence. Among the residues absolutely conserved between the now known HS enzymes, there is a set of amino acids at unique positions that control or greatly influence the size of the polymer that the enzyme can make.

Finally, using post-synthesis processing, larger molecular weight heparin can be degraded with specific glycosidases, ultrasonication, acids or a combination thereof to make lower molecular weight heparin/heparosan. This practice, however, is very difficult to achieve reproducibility and the heparin/heparosan must be meticulously repurified to remove the cleavage reagent and unwanted digestion products.

Structurally modified heparin/heparosan is no different conceptually than altering the size distribution of the heparin/heparosan product by changing particular amino acids in the desired HS and/or more particularly, but not limiting thereto, pmHS1 or pmHS2. Derivatives of UDP-GlcNAc, in which the acetyl group is missing from the amide (UDP-GlcN) or replaced with another chemically useful group (for example, phenyl to produce UDP-GlcNPhe), are expected to be particularly useful. The free amino group would be available for chemical reactions to derivatize heparin/heparosan in the former case with GlcN incorporation. In the latter case, GlcNPhe would make the polymer more hydrophobic or prone to making emulsions. The strong substrate specificity may rely on a particular subset of amino acids among the residues that are conserved. Specific changes to one or more of these residues creates a functional HS that interacts less specifically with one or more of the substrates than the native enzyme. This altered enzyme then utilizes alternate natural or special sugar nucleotides to incorporate sugar derivatives designed to allow different chemistries to be employed for the following purposes: (I) covalently coupling specific drugs, proteins, or toxins to the structurally modified heparin/heparosan for general or targeted drug delivery, radiological procedures, etc. (ii) covalently cross linking the heparin/heparosan itself or to other supports to achieve a gel, or other three dimensional biomaterial with stronger physical properties, and (iii) covalently linking heparin/heparosan to a surface to create a biocompatible film or monolayer.

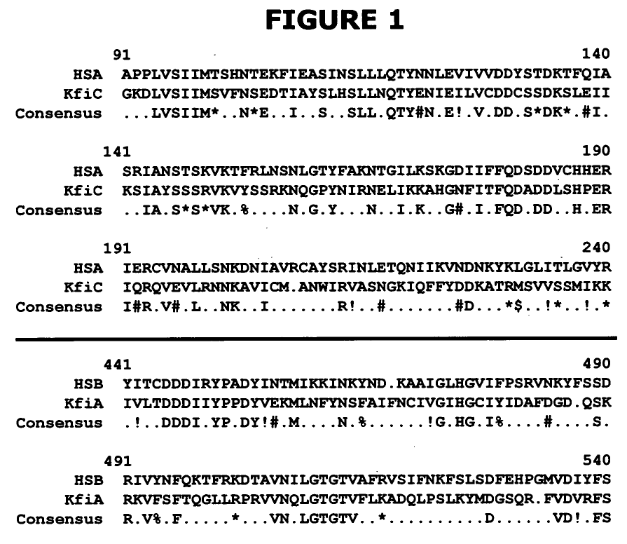

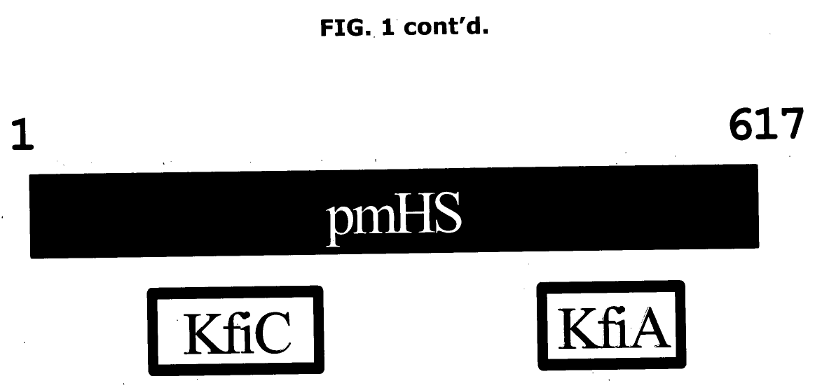

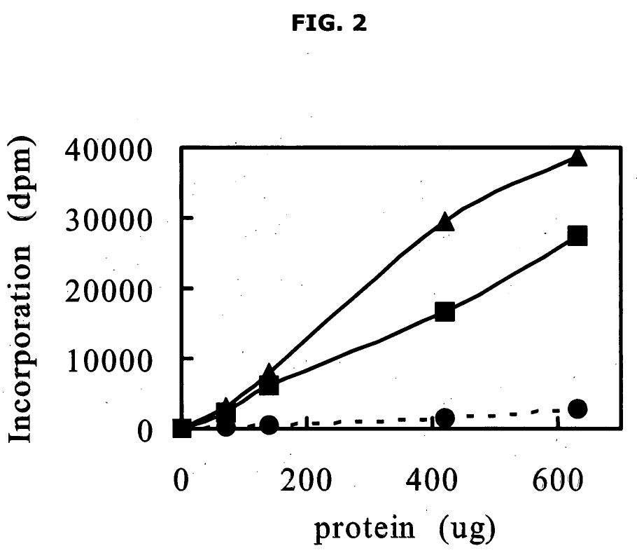

As stated hereinabove, Pasteurella multocida Type D, a causative agent of atrophic rhinitis in swine and pasteurellosis in other domestic animals, produces an extracellular polysaccharide capsule that is a putative virulence factor. It has been reported that the capsule of Type D was removed by treating microbes with heparin lyase III. A 617-residue enzyme, pmHS1, and a 651-residue enzyme, pmHS2, which are both authentic heparosan (unsulfated, unepimerized heparin) synthase enzymes have been molecularly cloned and are presently claimed and disclosed herein. Recombinant Escherichia coli-derived pmHS1 or pmHS2 catalyzes the polymerization of the monosaccharides from UDP-GlcNAc and UDP-GlcUA. Other structurally related sugar nucleotides do not substitute. Synthase activity was stimulated by the addition of an exogenous polymer acceptor. Large size molecules composed of many sugar residues were produced in vitro. The polysaccharide was sensitive to the action of heparin lyase III but resistant to hyaluronan lyase. The sequences of the pmHS1 and pmHS2 enzymes are not very similar to the vertebrate heparin/heparan sulfate glycosyltransferases, EXT½, or to other Pasteurella glycosaminoglycan synthases that produce hyaluronan or chondroitin. Certain motifs do exist, however, between the pmHS1, pmHS2, and KfiA and KfiC (see FIG. 1), thereby leading to deduced amino acid motifs that are conserved throughout this class of GAG synthases for the production of heparin/heparosan. The pmHS1 enzyme is the first microbial dual-action glycosyltransferase to be described that forms a polysaccharide composed of β4GlcUA-α4GlcNAc disaccharide repeats. In contrast, heparosan biosynthesis in E. coli K5 requires at least two separate polypeptides, KfiA and KfiC, to catalyze the same polymerization reaction.

Prior to recombinantly obtaining the pmHS1 gene and heterologously expressing it in a recombinant system, activity assays of P. multocida Type D enzymes were completed. Native membranes were prepared from a wild-type encapsulated Type D strain (P-3881; DeAngelis et al., 1996, the entirety of which is expressly incorporated herein in its entirety). The membranes were tested for in vitro sugar incorporation monitored by paper chromatography analysis. Characterization of the ability to co-polymerize the two sugars and utilize metal ions was performed. First, detection of co-polymerization activity of the Type D P. multocida strain was determined in vitro. The membranes plus UDP-[¹⁴C]GlcUA (300 μM; 1.5×10⁵ dpm) plus various combinations of the 2^{nd} sugar (UDP-GlcNAc, 900 μM) and/or EDTA chelator (45 mM) were mixed in 50 mM Tris, pH 7.2 with 20 mM MnCl₂ and 20 mM MgCl₂ reaction buffer. All reactions were performed at 30 degrees Celsius for 2.5 hours. The incorporation was measured by paper chromatography as disclosed in DeAngelis et al., 1996. The results of this co-polymerization activity are summarized in Table II.

TABLE II

| UDP-GlcNAc | EDTA | |
|---|---|---|
| Added? | Added? | Incorporation (dpm) |
| No | No | 520 |
| Yes | No | 9150 |
| No | Yes | 35 |
| Yes | Yes | 160 |

Thus, it is apparent that the Type D P. multocida strain P-3881 has a metal-dependent enzyme that copolymerized both heparin precursors into a polymer.

Second, the metal requirement of the Type D P. multocida HS activity was tested in vitro. Membranes plus UDP-[¹⁴C]GlcUA plus UDP-GlcNAc and buffer without the metals were mixed in a similar fashion as the preceding experiment except that various metals or EDTA (20 mM) were added as noted in Table III. The results of this metal specificity are summarized in Table III.

TABLE III

| Metal | dpm |
|---|---|
| None | 13 |
| Mg | 2960 |
| Mn | 3070 |
| Mn + Mg | 3000 |
| Co | 120 |

Thus, it is apparent that the Type D P. multocida HS requires either manganese or magnesium ion for enzymatic activity.

Further, the sugar specificity of the Type D P. multocida strain was determined in vitro in similar experiments. The ability to co-polymerize the sugars that compose the authentic backbone was tested by performing two parallel reactions:

UDP-[¹⁴C]GlcUA+various combinations of 2^{nd} UDP-sugars.

UDP-[³H]GlcNAc+various combinations of 2^{nd} UDP-sugars

The results of these experiments are summarized in Table IV. Significant ¹⁴C-GlcUA incorporation required UDP-GlcNAc and, conversely, significant ³H-GlcNAc incorporation required UDP-GlcUA; the enzyme copolymerizes the polysaccharide chain with both authentic heparin UDP-sugar precursors.

It should be added that the above-described results show that the native Type D P. multocida membrane enzymes have relaxed hexosamine transfer specificity in vitro. Such relaxed hexosamine transfer specificity is an advantage for syntheses where the UDP-sugar supplied can be manipulated. In such a manner, novel and non-naturally occurring polymers can be created. These novel, non-naturally occurring polymers have significant utility and novel biological properties.

TABLE IV
A. Hexosamine-transfer

| 2^{nd} Sugar Added | ¹⁴C dpm incorporation |
|---|---|
| None | 330 |
| UDP-GlcNAc | 2290 |
| UDP-GalNAc | 2790 |
| UDP-Glc | 450 |

B. Uronic Acid Transfer

| 2^{nd} Sugar Added | ³H dpm incorporation |
|---|---|
| None | 170 |
| UDP-GlcUA | 1000 |
| UDP-GalUA | 290 |
| UDP-Glc | 185 |

Isolation of HS Genes and Functional Testing

Expression of Recombinant P. multocida Heparosan Synthases—Portions of the pmHS1 ORF (normally 617 amino acids) was amplified from the various Type D genomic DNA template by 18 cycles of PCR with Taq polymerase. For constructing truncated enzymes, sense primers corresponding to residues 46 or 78 of the pmHS1 ORF plus an additional ATG at the 5′-end corresponding to a new Methionine residue start codon (thus creating pmHS 1^{K45M-617} (SEQ ID NOS:12 and 13 for nucleotide and amino acid sequences, respectively) or pmHS1^{I77M-617} (SEQ ID NOS:14 and 15 for nucleotide and amino acid sequences, respectively), respectively) were used. The sense primer used for creating pmHS1^{K45M-617} was ATGAATATAACACAATCAAAAAGTAATAAAATAG (SEQ ID NO:16), while the sense primer used for creating pmHS1^{I77M-617} was ATGAGCAATAGTGAATTAGGGATTACAAAAG (SEQ ID NO:17). For constructing the full-length enzyme (pmHS1¹⁻⁶¹⁷) for use as a control of membrane-associated insoluble catalyst, the sense primer (ATGAGCTTATTTAAACGTGCTACTGAGC (SEQ ID NO:18)) corresponded to the sequence at the deduced amino terminus of the native protein. In all three constructs, the same antisense primer (TTTACTCGTTATAAAAAGATAAACACGGAATAAG (SEQ ID NO:19)) encoding the carboxyl terminus including the stop codon was employed. The 651 amino acid pmHS2 ORF (open reading frame) predicted from a deposited Type A genome (gene annotated as “pglA”; Univ. of Minnesota genome project was amplified from Type A (P-1059), Type D (P-3881) or Type F (P-4218) genomic DNA template by 18 cycles of PCR (94° C., 30 s; 72° C., 2.5 min; 52° C.; 30 s) with Taq DNA polymerase (Fisher). For constructing the full-length enzyme, the sense primer (ATGAAGAGAAAAAAAGAGATGACTC) (SEQ ID NO:20)) corresponded to the sequence at the deduced amino terminus of the ORF beginning with the start codon and the antisense primer (ATCATTATAAAAAATAAAAAGGTAAACAGG) (SEQ ID NO:21)) encoded the carboxyl terminus including the stop codon.

The amplicons were cloned using the pETBlue-1 Acceptor system (Novagen) according to the manufacturer's instructions. The Taq-generated single A overhang is used to facilitate the cloning of the open reading frame downstream of the T7 promoter and the ribosome binding site of the vector. The ligated products were transformed into E. coli NovaBlue and plated on LB carbenicillin (50 pg/ml) and tetracycline (13 pg/ml) under conditions for blue/white screening. White colonies were analyzed by PCR-based screening and by restriction digestion. Plasmids with the desired ORF were transformed into E. coli Tuner, the T7 RNA polymerase-containing expression host, and maintained on LB media with carbenicillin and chloramphenicol (34 μg/ml) at 30° C. Mid-log phase cultures were induced with β-isopropylthiogalactoside (0.2 mM final) for 5 hrs. The cells were harvested by centrifugation, frozen, and membranes were prepared according to a cold lysozyme/sonication method except 0.1 mM mercaptoethanol was included during the sonication steps. The supernatant was kept as the source of soluble molecules while the membrane proteins were found in the pellets which were suspended in 50 mM Tris, pH 7.2, 0.1 mM EDTA and protease inhibitors. Assays for Heparosan Synthase and Single-Action Catalyst Activity—Incorporation of radiolabeled monosaccharides from UDP-[¹⁴C]GlcUA and/or UDP-[³H]GlcNAc precursors (NEN) was used to monitor heparosan synthase activity (i.e. polymerization of long chains). Samples were assayed in a buffer containing 50 mM Tris, pH 7.2, 10 mM MgCl2, 10 mM MnCl₂, 0-0.6 mM UDP-GlcUA, and 0-0.6 mM UDP-GlcNAc at 30° C. Depending on the experiment, a Type D acceptor polymer processed by extended ultrasonication of a capsular polysaccharide preparation (isolated by cetylpyridinium chloride precipitation of the spent Type D culture media) was also added to the reaction mixture. For single action activity assays, similar conditions were employed except that only one type of UDP-sugar (either UDP-GlcUA or UDP-GlcNAc in appropriate radioactive form) was employed in reactions with acceptor polysaccharide; therefore the addition of a single sugar was readily detectable. The reaction products were separated from substrates by descending paper (Whatman 3M) chromatography with ethanol/1 M ammonium acetate, pH 5.5, development solvent (65:35). The origin of the paper strip was cut out, eluted with water, and the incorporation of radioactive sugars into polymer was detected by liquid scintillation counting with BioSafe II cocktail (RPI).

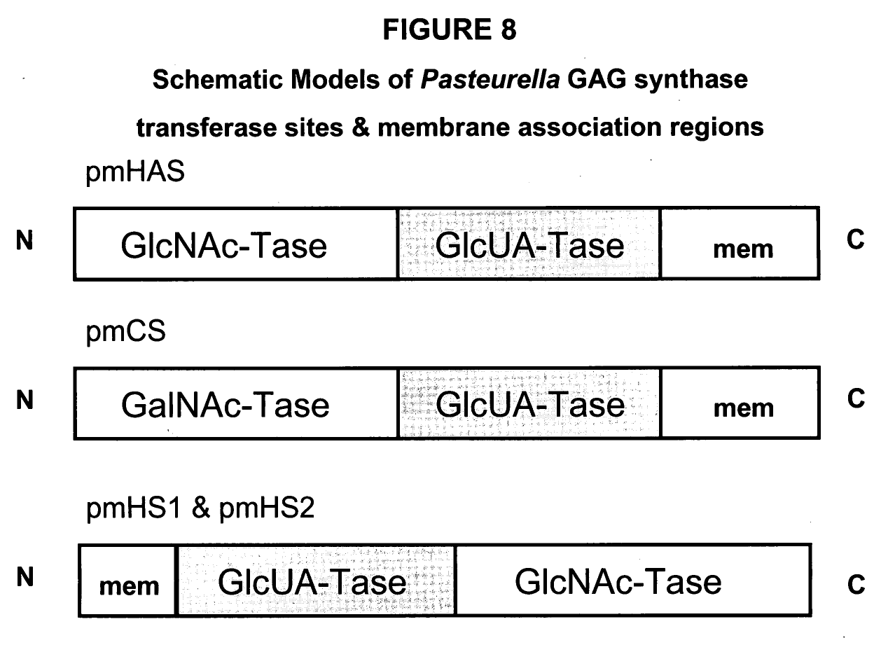

Heparosan synthase activity (polymerization of long chains in presence of both UDP-sugar precursors) of the truncated enzymes was measured in the supernatant (soluble) and membrane fractions. As seen in Table V, removal of portions of the amino terminus confers solubility to the pmHS1 catalyst while retaining activity. This result could not be predicted with existing information; in fact, the finding is opposite to the pmHAS hyaluronan synthase that requires carboxyl terminus truncation to achieve solubility (Jing and DeAngelis, Glycobiology, 2000 (see FIG. 8)). Furthermore, the existing computer programs for predicting transmembrane segments or membrane associations or hydrophobicity plots (as encompassed in programs at the WWW site http://us.expasy.org) do not predict that the amino terminal region is the membrane association region; the finding was determined empirically. The pmHS2, which is a pmHS1 homolog, should be amenable to the same genetic engineering procedure as pmHS1, thus creating another soluble catalyst. Therefore, soluble forms of pmHS2 are also within the scope of the presently disclosed and claimed invention.

TABLE V
Heparosan Synthase Activity of Wild-type and truncated pmHS1

| | Activity | |
|---|---|---|
| Truncation | (14C-GlcUA dpm) | Fraction |
| full length pmHS1¹⁻⁶¹⁷ | 100 | Soluble |
| | 15,000 | Membrane |
| pmHS1^{I77M-617} | 11,000 | Soluble |
| (Δ 77AA of N terminus) | 700 | Membrane |
| pmHS1^{K45M-617} | 1,600 | Soluble |
| (Δ 45AA of N terminus) | 180 | Membrane |

Design of Single Action Mutants. Comparisons of the two known sets of heparin/heparosan biosynthesis enzymes from the E. Coli K5 Kfi locus (GenBank Accession Number X77617), the pmHS2 enzyme, and the pmHS1 from Type D capsular locus, allows for the initial assessment and bioinformatic prediction of new enzymes based on the amino acid sequence data. The closer the match (% identity) in a single polypeptide for the two sequence motifs described hereinafter (corresponding to the critical elements of the GlcUA-transferase and the GlcNAc-transferase), the higher the probability that the query enzyme is a new heparin/heparosan synthase (a single dual-action enzyme). The closer the match (% identity) in two polypeptides (especially if encoded in the same operon or transcriptional unit) for the two sequence motifs, the higher the probability that the query enzymes are a pair of single-action glycosyltransferases. Thus, one of ordinary skill in the art would appreciate that given the following motifs, one would be able to ascertain and ascribe a probable heparin synthase function to a newly discovered enzyme and then test this ascribed function in a manner to confirm the enzymatic activity. Thus, single dual-action enzymes possessing enzymatic activity to produce heparin/heparosan and having at least one of the two disclosed motifs are contemplated as being encompassed by the presently claimed and disclosed invention.

Motif I: (SEQ ID NO: 22)
QTYXN(L/I)EX₄DDX(S/T)(S/T)D(K/N)(T/S)X₆IAX(S/T)
(S/T)(S/T)(K/R)V(K/R)X₆NXG
XYX₁₆FQDXDDX(C/S)H(H/P)ERIXR
Motif II: (SEQ ID NO: 23)
(K/R)DXGKFIX₁₂₋₁₇DDDI(R/I)YPXDYX₃MX₄₀₋₅₀VNXLGTGTV

Motif I corresponds to the GlcUA transferase portion of the enzyme, while Motif II corresponds to the GlcNAc transferase portion of the enzyme. With respect to the motifs:

X=any residue

parentheses enclose a subset of potential residues [separated by a slash] that may be at a particular position (e.g.—(K/R) indicates that either K or R may be found at the position, that is, there are semiconserved residues at that position).

The consensus X spacing is shown with the number of residues in subscript (e.g. X₁₂₋₁₇), but there are weaker constraints on these particular residues; thus, spacing may be longer or shorter. Conserved residues may be slightly different in a few places, especially if a chemically similar amino acid is substituted (e.g. K for a R, or E for a D). Overall, at the 90% match level, the confidence in this predictive method is very high, but even a 70-50% match level without excessive gap introduction (e.g. altered spacing between conserved residues) or rearrangements (miss-positioning with respect to order of appearance in the amino to carboxyl direction) would also be considered to be within the scope of these motifs. One of ordinary skill in the art, given the present specification, general knowledge of the art, as well as the extensive literature of sequence similarity and sequence statistics (e.g. the BLAST information website at www.ncbi.nlm.mih.gov) would appreciate the ability of a practitioner to identify potential new heparin/heparosan synthases based upon sequence similarity or adherence to the motifs presented herein and thereafter test for functionality by means of heterologous expression, to name but one example.

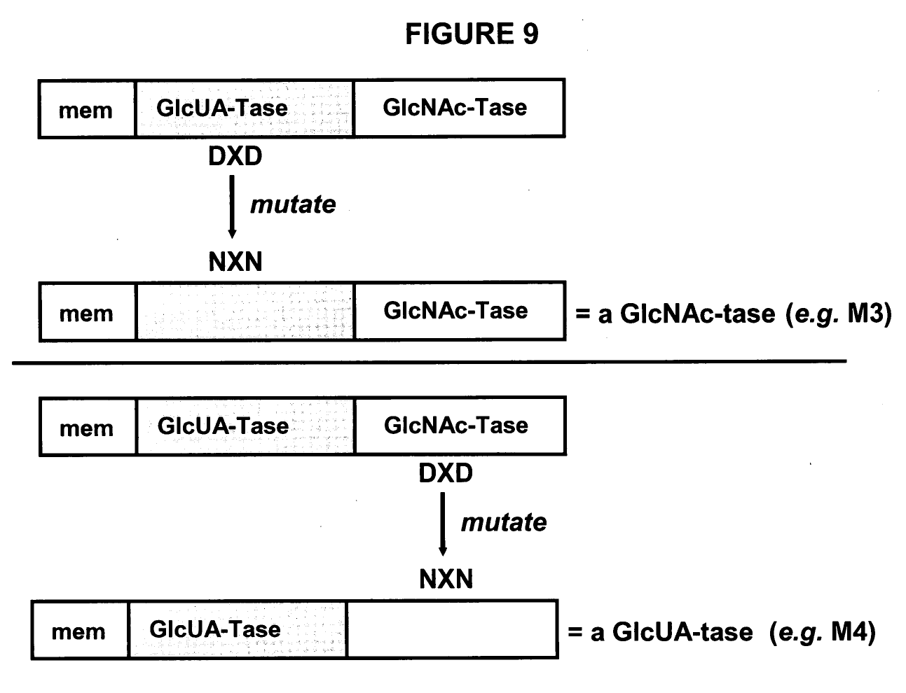

The disclosed motifs or domains contain various critical residue motifs that may be changed (mutated) or dissected from the original, naturally occurring protein, and function will be affected; removal of critical residues will perturb the important action of the remaining critical motifs or domains. The pmHS1 and pmHS2 enzymes in the natural state are dual action enzymes with two separate active sites or domains. Since the sites are relatively functionally independent, the alteration of one site or domain does not destroy the activity of the other unmutated site. Such is the case with mutated, soluble versions of pmHS1 (such as thioredoxin fusions containing full-length pmHS1-D¹⁸¹N-D¹⁸³N [data code=M3] (SEQ ID NO:25) or pmHS1-D⁴⁴⁴N-D⁴⁴⁶N [data code=M4] (SEQ ID NO:27)) are shown and described herein and falls within the scope of the presently claimed and disclosed invention of single action transferases (see FIG. 9).

The recombinant wild-type pmHS1 enzyme fused with thioredoxin protein at the amino terminus (using the standard protocols and procedures for the pBAD/Thio-TOPO kit of Invitrogen, Inc) is still functional as a dual action synthase. Furthermore, the enzyme is a more soluble enzyme (found in supernatant of 100,000×g spin after lysis) that is expressed at higher levels than full-length native sequence pmHS1 which is a membrane protein (found in pellet of 100,000×g spin after lysis (see Table V)). Mutations at either HS motif described above were made using the standard procedures of the Stratagene QuickChange site-directed mutagenesis kit (Stratagene) with the appropriate oligonucleotides encoding the desired amino acid substitutions; the selected D×D submotifs were mutated to N×N (changes both negative acidic Glu residues to neutral Asn residues). The sense primer used for creating GlcNAc-Tase was ATATTATTTTCTTTCAGAATAGCAATGATGTATGTCACCATG (SEQ ID NO: 28), and the antisense primer was CATGGTGACATACATCATTGCTATTCTGAAAGAAAATAATAT (SEQ ID NO:29). The sense primer used for creating GlcUA-Tase was GATATTATATAACTTGTAATGATAATATCCGGTATCC (SEQ ID NO: 30), and the antisense primer was GGATACCGGATATTATCATTACAAGTTATATAATATC (SEQ ID NO:31). Sugar transfer activity analyses for heparosan production (dual action catalyst), GlcNAc addition (single action catalyst) or GlcUA addition (single action catalyst) were performed. The data show that neither N×N mutants can polymerize heparosan (Table VI) but the mutants will add on a single sugar (Tables VII and VIII).

TABLE VI
Heparosan Dual Action Activity Assay of
Recombinant Thio-fusion pmHS1 mutants.

| enzyme | 3H-GlcNAc (dpm) | 14C-GlcA (dpm) |
|---|---|---|
| M3-2 | 57 | 18 |
| M3-4 | 88 | 120 |
| M4-1 | 58 | 140 |
| M4-2 | 67 | 160 |
| M4-3 | 78 | 160 |
| M4-4 | 39 | 150 |
| Wild type | 34,000 | 73,000 |
| Vector alone | 30 | 19 |

(note: M3 and M4 are pmHS1- D¹⁸¹N-D¹⁸³N and pmHS1-D⁴⁴⁴N-D⁴⁴⁶N mutants, respectively. The second numeral [−X] signifies the independent clone number)

TABLE VII
GlcUA-transferase (Single Action) Activity Assay of
Recombinant Thio-fusion pmHS1 mutants.

| enzyme | 14C-GlcUA (dpm) |
|---|---|
| M4-1 | 9,100 |
| M4-2 | 7,600 |
| M4-3 | 6,700 |
| M4-4 | 2,400 |
| Wild type | 15,000 |
| Vector alone | 70 |

TABLE VIII
GlcNAc-transferase (Single Action) Activity Assay of
Recombinant Thio-fusion pmHS1.

| enzyme | ³H-GlcNAc (dpm) |
|---|---|
| M3-2 | 1,900 |
| M3-4 | 2,800 |
| Wild type | 41,000 |
| Vector alone | 270 |

The first generation mutants express at different levels (as assessed by SDS-PAGE followed by Western blot), thus the absolute signals are not always equivalent to wild-type enzyme, but the mutants retain authentic single action activity. Therefore, these new catalysts (i.e. GlcNAc-transferase=pmHS1-D¹⁸¹N-D¹⁸³N (SEQ ID NO:25) and GlcUA-transferase=pmHS1-D⁴⁴⁴N-D⁴⁴⁶N (SEQ ID NO:27)) are more useful for the step-wise synthesis of GAG polymers. Of course, the appropriate mutation of other residues in the motifs or any residue critical for function will result in enzymes that are single-action catalysts if one site or domain is inactivated and the other site or domain is preserved.

Modification of Heparosan. Bacteria-derived heparosan may be converted by epimerization and sulfation into a polymer that resembles the mammalian heparin and heparan sulfate because all the modifying enzymes have been identified. In general, sulfation with chemical reagents (SO₃, chlorosulfonic acid) or sulfo-transferases (i.e. 2-0-GlcUA-sulfotransferase, etc.) and PAPs precursor is possible. N-sulfation can be done by using either chemical means (hydrazinolysis and subsequent N-sulfation) or enzymatic means with dual function deacetylase/N-sulfotransferase. For creation of iduronic acid, epimerization can be performed enzymatically with heparin epimerase or chemically with super-critical carbon dioxide. The art is replete with articles, methods, and procedures for sulfating and epimerizing heparosan to form heparin. Thus, given the present specification which discloses and teaches methods for the recombinant production of Heparosan, one of ordinary skill in the art would be capable of producing Heparin therefrom. As such, Heparin obtained through the process of sulfating and epimerizing Heparosan is contemplated as falling within the scope of the presently disclosed and claimed invention.

### New Heparin/Heparosan Synthase and Characterization

pmHS1 or pmHS2 (or an improved recombinant version) may be more economical and useful sources of heparosan than E. coli K5 for several reasons. pmHS1 and pmHS2 have a higher intrinsic biosynthetic capacity for capsule production. The Pasteurella capsule radius often exceeds the cell diameter when observed by light microscopy of India Ink-prepared cells. On the other hand, visualization of the meager E. coli K5 capsule often requires electron microscopy. From a safety standpoint, E. coli K5 is a human pathogen, while Type D Pasteurella has only been reported to cause disease in animals. Furthermore, with respect to recombinant gene manipulation to create better production hosts, the benefits of handling only a single gene encoding pmHS1 or pmHS2, which are dual action syntheses, in comparison to utilizing KfiA and C (and probably KfiB) are obvious. The in vitro properties of pmHS1 and pmHS2 are also superior; these enzymes can make large chains in vitro either with or without an exogenous acceptor sugar, but KfiA and KfiC do not.

Immunological Analysis of Recombinant pmHS1 and pmHS2. The pmHS1 (derived from Type D P-4058) and pmHS2 (derived from Type D P-3881) polypeptides in membrane preparations and extracts were analyzed using standard 8% polyacrylamide SDS gels and Western blotting utilizing a monospecific antibody directed against a synthetic peptide (acetyl-KGDIIFFQDSDDVCHHERIER-amide) (SEQ ID NO:32)) corresponding to residues 173 to 193 of pmHS1 or residues 207 to 227 of pmHS2 using colorimetric detection methodology. Total cell extracts were made by suspending the cell pellet from logarithmic phase cultures in 1× gel sample buffer, boiling for 2 min, and clarifying by centrifugation.

Assays for Heparosan Synthase Activity. Incorporation of radiolabeled monosaccharides from UDP-[14C]GlcUA and/or UDP-[3H]GlcNAc precursors (Perkin Elmer NEN) was used to monitor heparosan synthase activity. The metal preference of pmHS2 was assessed by comparing the signal from a “no metal” control reaction (0.5 mM EDTA) to reactions containing 10 to 20 mM manganese, magnesium, or cobalt chloride. To test the sugar transfer specificity of pmHS2, various UDP-sugars (UDP-GalNAc, UDP-GalUA (galacturonic acid), or UDP-Glc (glucose)) were substituted for the authentic heparosan precursors. The data from the recombinant construct containing pmHS2 gene from the Type D strain P-3881 are presented, but the results were similar to experiments with constructs derived from the Type A strain P-1059.

Size Analysis and Enzymatic Degradation of Labeled Polymers. Gel filtration chromatography was used to analyze the size distribution of the labeled polymers. Separations were performed with either a Polysep-GFC-P 4000 column or Polysep-GFC-P 5000 column (300′7.8 mm; Phenomenex) eluted with 0.2 M sodium nitrate at 0.6 ml/min. Radioactivity was monitored with an in-line Radioflow LB508 detector (EG & G Berthold; 500 ml flow cell) using Unisafe I cocktail (1.8 ml/min; Zinsser). The column was standardized with fluorescein-labeled dextrans of various sizes. To further characterize the radiolabeled polymers, depolymerization tests with specific glycosidases were performed (Flavobacterium heparin lyase III or Streptomyces HA lyase).

Southern Blot Analyses of pmHS1 and pmHS2 genes. A specific hybridization probe for each P. multocida heparosan synthase isozyme was generated. DNA was excised from the ORFs of the pETBlue-1 expression plasmids (the 1.3 kb fragment of NcoI and XhoI double-cut pmHS2 derived from strain P-1059; the 701 bp fragment of EcoRI-cut pmHS1 derived from strain P-4058, gel-purified, and used to generate digoxigenin-labeled probes utilizing the manufacturer guidelines (High Prime system, Boehringer Mannheim)). Typical Southern blot methodology was performed on HinDIII or NcoI/XhoI-cut genomic DNA from P. multocida Type A (P-1059) or D (P-3881 and P-4058) genomic DNAs. Duplicate nitrocellulose replicas were screened by hybridization (DIG Easy Hyb, 40° C.; 16 hrs) with either the pmHS1 or the pmHS2 digoxigenin-labeled probe and colorimetric development.

GenBank Deposits of pmHS2 Sequences: The sequences of a pmHS2 clone from a Type A and a Type D strain were deposited in GenBank (AY292199 and AY292200, respectively). These sequences are ˜99% identical to the deposited genome sequence.

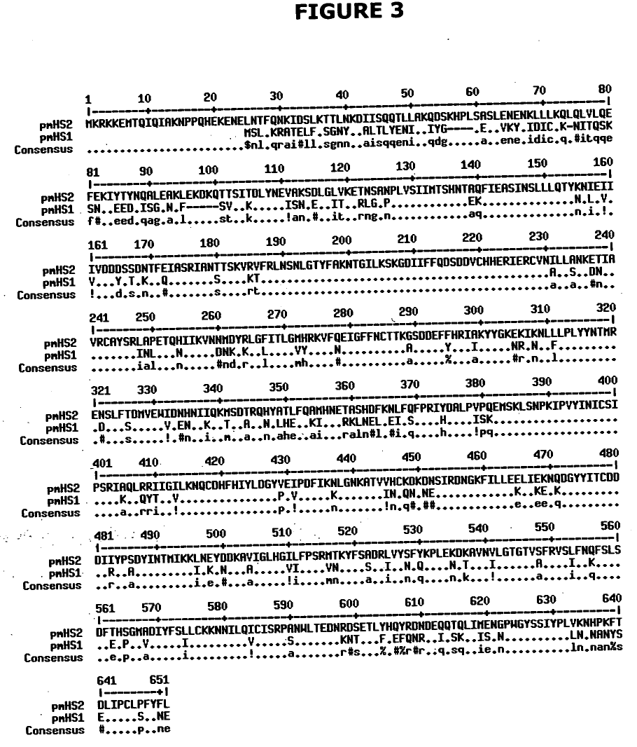

The pmHS1 and pmHS2 Nomenclature. A deduced gene was recently uncovered by the University of Minnesota in their Type A P. multocida genome project, called pglA (GenBank Accession Number AAK02498), encoding 651 amino acids which is also similar to pmHS (approximately 73% identical in the major overlapping region; FIG. 3). However, the pglA gene is not located in the putative capsule locus with the pmHS. This research group did not establish the function of pglA, but this name has been used to describe another product involved in bacterial protein glycosylation from Campylobacter jejuni. The pglA-like genes were cloned from several P. multocida capsular types, and nearly identical DNA sequences were found. It is shown below that the Pasteurella PglA enzyme is actually a functional heparosan synthase, and therefore it is proposed that pmHS2 is a more appropriate nomenclature. Thus the original pmHS enzyme should now be called pmHS1.

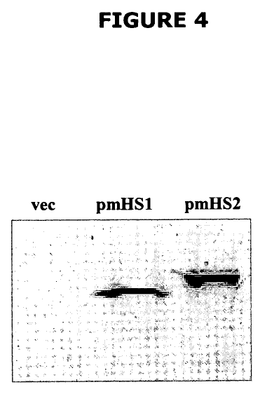

Heterologous Expression and Characterization of Functional P. multocida Heparosan Synthases. Both recombinant pmHS1 and pmHS2 were prepared in an E. coli host that does not normally produce GAG polymers. The two recombinant proteins were detected by Western blotting (FIG. 4); as predicted from the deduced ORF sequence, the larger pmHS2 polypeptide migrates slower than pmHS1 on SDS-PAGE. Attempts to visualize the native pmHS2 protein from several P. multocida isolates in various media (including defined or complex media supplemented with chicken tissue or sera) were unsuccessful. Native pmHS1 from Type D strains, however, was easily detected in parallel tests; the protein migrated identically to recombinant pmHS1 (not shown).

Membrane extracts derived from E. coli Tuner cells containing the plasmid encoding pmHS2, but not samples from cells with the vector alone, synthesized polymer in vitro when supplied with both UDP-GlcUA and UDP-GlcNAc simultaneously (Table IX). No substantial incorporation of radiolabeled GlcUA into polymer was observed if UDP-GlcNAc was omitted, or if UDP-GalNAc or UDP-Glc was substituted for UDP-GlcNAc. Conversely, in experiments using radiolabeled UDP-GlcNAc, substantial incorporation of label into polymer was only noted when UDP-GlcUA was also present; UDP-GalUA or UDP-Glc did not substitute for UDP-GlcUA. The identity of the pmHS2-derived polymer as heparosan was verified by its sensitivity to Flavobacterium heparin lyase III (99.5% polymer destroyed) and its resistance to the action of Streptomyces HA lyase. Therefore, pmHS1 and pmHS2 are both selective glycosyltransferases that catalyze the production of authentic heparosan polymer.

The maximal activity of pmHS2 was observed in reactions that contained Mn2+ ion, but Mg2+ and Co2+ also supported incorporation (approximately 25%-30% of level with Mn2+). On the other hand, the level of pmHS1 activity was very similar in the presence of Mn2+ or Mg2+.

TABLE IX
Transferase Specificity of Recombinant pmHS2 for Sugar
Nucleotides. Crude membranes from cells with plasmid
encoding the Type D pmHS2 enzyme (360 μg of total
protein), or no insert, vector (360 μg of total protein),
were incubated in 50 μl of assay buffer for 120 min
either with A, UDP-[³H]GlcNAc, or B, UDP-[³H]GlcUA.
The radiolabeled sugar (500 μM; 0.4 μCi/reaction)
was used in the presence of the indicated
second unlabeled sugar nucleotide (500 μM).
The incorporation into polymer was assessed
by paper chromatography. A representative experiment is
shown. The recombinant pmHS incorporated only the
authentic heparin precursors into polysaccharide.

[³H]GlcNAc
Incorporation
(DPM)

| A. | 2^{nd} Sugar | pmHS2 | vector |
|---|---|---|---|
| | none | 5,200 | 450 |
| | UDP-GlcUA | 72,000 | 400 |
| | UDP-GalUA | 4,100 | 430 |
| | UDP-Glc | 4,200 | 400 |

[³H]GlcUA
Incorporation
(DPM)

| B. | 2^{nd} Sugar | pmHS2 | vector |
|---|---|---|---|
| | none | 450 | 140 |
| | UDP-GlcNAc | 110,000 | 160 |
| | UDP-GalNAc | 430 | 170 |
| | UDP-Glc | 1,800 | 140 |

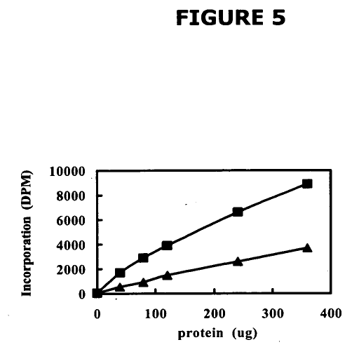

The addition of the heparosan polymer acceptor only increased pmHS2-catalyzed sugar incorporation by about 2.5-fold (FIG. 5). In contrast, pmHS1 was stimulated at least 7- to 25-fold in comparison to parallel reactions without acceptor in analogy to observations of pmHAS and pmCS (ref. #14 and #18). The acceptor stimulation of activity appears to be due to the lower efficiency or slower rate of initiation of a new polymer chain in comparison to the elongation stage in vitro. The exogenous acceptor sugar associates with the recombinant enzyme's binding site for the nascent chain and then is elongated rapidly.

Analysis by gel filtration chromatography indicated that recombinant pmHS1 produced long polymer chains (˜200 to 600 kDa based on dextran standards) in vitro without acceptor (FIG. 6B). If acceptor polymer was supplied to parallel reaction mixtures with pmHS1, then high levels of sugar incorporation are observed as short chains added onto the acceptor. The pmHS2 enzyme made shorter chains (broad peak centered on ˜330 kDa corresponding to ˜1.7×10e3 monosaccharides; FIG. 6A) than pmHS1 under identical conditions in vitro. The pmHS2 also catalyzed the extension of the exogenously supplied acceptor chains, but the effect was not as dramatic as pmHS1.

Overall, pmHS1 and pmHS2 are both heparosan synthases with similar amino acid sequences, but their metal cofactor specificity, acceptor usage, and polymer product size distributions are distinct.

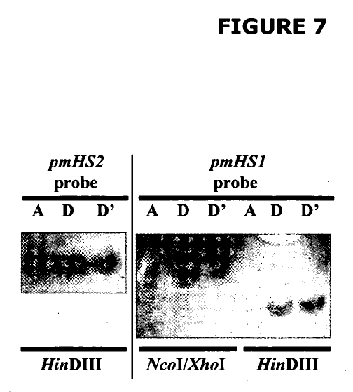

The pmHS2 gene is not in the capsule locus. Several capsule loci of different P. multocida types have been reported. The pmHAS, pmHS1 or pmCS synthase genes of Types A, D, or F strains, respectively, are adjacent to a gene encoding UDP-glucose dehydrogenase in the capsule locus. Flanking these two genes are various putative transporter genes. However, the pmHS2 gene in the Type A genome (strain pm7O; resides between two metabolic genes not known to be involved in directly involved in capsule polymer synthesis, alanine racemase (alr) and glucose-6-phosphate isomerase (pgi). It was found that pmHS2 in several tested strains was flanked by the same two genes by sequencing PCR amplicons derived from genomic DNA. Southern blotting was used to show that the pmHS1 and pmHS2 are also unlinked in the tested Type D strains in our collection (FIG. 7). The experiment also shows that the pmHS1 gene is not found in the HA-producing Type A strain.

Thus, it should be apparent that there has been provided in accordance with the present invention purified nucleic acid segments having coding regions encoding enzymatically active dual-action, single-action and soluble heparin/heparosan synthases, methods of producing heparin/heparosan from the pmHS1 or pmHS2 gene or mutants or fragments thereof, and the use of heparin/heparosan produced therefrom, that fully satisfies the objectives and advantages set forth above. Although the invention has been described in conjunction with specific embodiments thereof, it is evident that many alternatives, modifications, and variations will be apparent to those skilled in the art. For example, in general, any molecular genetic or biochemical modification (i.e. soluble or single-action catalyst generation) that is useful for pmHS1 will be applicable to pmHS2. Accordingly, it is intended to embrace all such alternatives, modifications, and variations that fall within the spirit and broad scope of the appended claims.

### References

The following references, to the extent that they provide exemplary procedural or other details supplementary to those set forth herein, are specifically incorporated herein by reference.

1. Roden, L. (1980) in The Biochemistry of Glycoproteins and Proteoglycans (Lennarz, W. J., ed) pp. 267-371, Plenum Publishing Corp., New York

2. Lidholt, K. (1997) Biochem. Soc. Trans. 25, 866-870

3. Esko, J. D. and Lindahl U. (2001) J. Clin. Invest.,108:169-173

4. Roberts, I. S. (1996) Annu. Rev. Microbiol. 50, 285-315

5. Vann, W. F., Schmidt, M. A., Jann, B., and Jann, K. (1981) Eur. J. Biochem. 116, 359-364.

6. Rodriguez, M. L., Jann, B., and Jann, K. (1988) Eur. J. Biochem. 177, 117-124

7. Griffiths, G., Cook, N. J., Gottfridson, E., Lind, T., Lidholt, K., and Roberts, I. S. (1998) J. Biol. Chem., 273,11752-11757.

8. Hodson, N., Griffiths, G., Cook, N., Pourhossein, M., Gottfridson, E., Lind,T., Lidholt, K. and Roberts, I. S. (2000) J. Biol. Chem., 275:27311-27315.

9. Rimler, R. B. (1994) Vet. Rec. 134, 191-192.

10. Rimler, R. B., Register, K. B., Magyar, T., and Ackermann, M. R. (1995) Vet. Microbiol. 47, 287-294.

11. Rosner, H., Grimmecke, H. D., Knirel, Y. A., and Shashkov, A. S. (1992) Carb. Res. 223, 329-333

12. DeAngelis, P. L., Jing, W., Drake, R. R. and Achyuthan, A. M. (1998) J. Biol. Chem. 273, 8454-8458

13. Rimler, R. B. and Rhoades, K. R. (1987) J. Clinic. Microbiol. 25, 615-618

14. DeAngelis, P. L. and Padgett-McCue, A. J. (2000) J. Biol. Chem., 275: 24124-24129.

15. Townsend, K. M., Boyce, J. D., Chung, J. Y., Frost, A. J., and Adler, B. (2001) J. Clin. Microbiol. 39:924-929

16. DeAngelis, P. L., and Weigel, P. H. (1994) Biochem. 33, 9033-9039

17. May, B. J., Zhang, Q., Li, L. L., Paustian, M. L., Whittam, T. S., and Kapur, V. (2001) Proc. Natl. Acad. Sci. U.S.A. 98:3460-3465

18. DeAngelis, P. L. (1999) J. Biol. Chem.274, 26557-26562

19. Jing, W. and DeAngelis, P. L. (2000) Glycobiology 10: 883-889

20. Duncan, G., McCormick, C., and Tufaro, F. (2001) J. Clin. Invest.108: 511-516

21. Corpet, F. (1988) Nucleic Acids Res. 16:10890 18

22. Berrington, A. W., Y. C. Tan, Y. Srikhanta, B. Kuipers, P. van der Ley, I. R. Peak, and M. P. Jennings. (2002) FEMS Immunol Med Microbiol. 34:267-75.

23. Biosca, E. G., H. Llorens, E. Garay, and C. Amaro. (1993) Infect Immun. 61:1611-8.

24. DeAngelis, P. L. (2002) Glycobiology 12:9R-16R.

25. DeAngelis, P. L., N. S. Gunay, T. Toida, W. J. Mao, and R. J. Linhardt. (2002) Carbohydr Res. 337:1547-52.

26. DeAngelis, P. L., and C. L. White. (2002) J Biol Chem 277:7209-13.

27. Jing, W., and P. L. DeAngelis. (2003) Glycobiology 13:661-71.

28. Pandit, K. K., and J. E. Smith. (1993) Res Vet Sci 54:20-4.

29. Saunders, N. J., A. C. Jeffries, J. F. Peden, D. W. Hood, H. Tettelin, R. Rappuoli, and E. R. Moxon. (2000) Mol Microbiol 37:207-15.

30. Sellin, M., S. Hakansson, and M. Norgren. (1995). Microb Pathog 18:401-15.

31. Pummill, P. E. and P. L. DeAngelis. (2003) J. Biol. Chem. 278:19808-19814.

32. DeAngelis, P. L.(1996) Biochemistry. 35:9768-9771.

33. DeAngelis, P. L. and A. M. Achyuthan. (1996) J. Biol. Chem. 271:23657-23660.

What I claim is:

1. A purified nucleic acid segment comprising at least one of:
(A) a coding region encoding enzymatically active, soluble heparin synthase;
(B) a purified nucleic acid segment encoding an enzymatically active, soluble heparin synthase isolated from Pasteurella multocida;
(C) a purified nucleic acid segment encoding the soluble heparin synthase of SEQ ID NO:13 or 15;
(D) a purified nucleic acid segment encoding an enzymatically active, soluble heparin synthase, wherein the enzymatically active, soluble heparin synthase is at least 70% identical to SEQ ID NO:13 or 15;
(E) a purified nucleic acid segment comprising a nucleotide sequence in accordance with SEQ ID NO:12 or 14;
(F) a purified nucleic acid segment capable of hybridizing to the nucleotide sequence of SEQ ID NO:12 or 14 under low, medium or high stringency conditions;
(G) a purified nucleic acid segment having semiconservative or conservative amino acid changes or being a truncated segment when compared to the nucleotide sequence of SEQ ID NO:12 or 14;
(H) a purified nucleic acid segment having at least one nucleic acid segment sufficiently duplicative of the nucleic acid segment in accordance with SEQ ID NO:12 or 14 to allow possession of the biological property of encoding for a soluble Pasteurella multocida heparin synthase;
(I) a purified nucleic acid segment encoding an enzymatically active, soluble heparin synthase, wherein the enzymatically active, soluble heparin synthase is a fragment of SEQ ID NO:2, 4, 6, 13, 15 or 34; and
(J) a purified nucleic acid segment comprising a fragment of a nucleic acid sequence in accordance with SEQ ID NO:1, 3, 5, 12, 14 or 33, and wherein the purified nucleic acid segment encodes an enzymatically active, soluble heparin synthase.

2. The purified nucleic acid segment of claim 1 wherein the purified nucleic acid segment is provided in a recombinant vector selected from the group consisting of a plasmid, cosmid, phage, integrated cassette or virus vector.

3. The purified nucleic acid segment of claim 2, wherein the recombinant vector containing the purified nucleic acid segment is used to electroporate, transform or transduce a host cell to produce a recombinant host cell having the recombinant vector.

4. The purified nucleic acid segment of claim 3, wherein the recombinant host cell produces heparin.

5. The purified nucleic acid segment of claim 3, wherein the recombinant host cell produces heparin synthase.

6. The purified nucleic acid segment of claim 3, wherein the enzymatically active heparin synthase is capable of producing a heparin polymer having a modified structure or modified size distribution.

7. The purified nucleic acid segment of claim 3 wherein the recombinant host cell further comprises at least one of an epimerase, a sulfotransferase, and combinations thereof.

8. A method for producing a heparin polymer in vitro comprising the steps of:
providing a soluble heparin synthase;
placing the soluble heparin synthase in a reaction mixture containing UDP-GlcNAc and UDP-GlcUA and at least one divalent metal ion suitable for the synthesis of a heparin polymer; and
extracting the heparin polymer out of the reaction mixture.

9. The method of claim 8 wherein, in the step of providing the soluble heparin synthase, the soluble heparin synthase is encoded by the purified nucleic acid segment of claim 1.

10. A purified nucleic acid segment comprising at least one of:
(A) a coding region encoding a modified heparin synthase, wherein the modified heparin synthase is capable of adding at least one of GlcUA and GlcNAc to a heparin polymer;
(B) a coding region encoding a modified soluble heparin synthase, wherein the modified soluble heparin synthase is capable of adding at least one of GlcUA and GlcNAc to a heparin polymer;
(C) a purified nucleic acid segment encoding a modified heparin synthase of SEQ ID NO:25 or 27 wherein the modified heparin synthase is capable of adding at least one of GlcUA and GlcNAc to a heparin polymer;
(D) a purified nucleic acid segment encoding a modified heparin synthase having at least about 70% identity to SEQ ID NO:25 or 27 and wherein the modified heparin synthase is capable of adding at least one of GlcUA and GlcNAc-to a heparin polymer;
(E) a purified nucleic acid segment comprising a nucleotide sequence in accordance with SEQ ID NO:24 or 26;
(F) a purified nucleic acid segment capable of hybridizing to the nucleotide sequence of SEQ ID NO:24 or 26 under low, medium or high stringency conditions;
(G) a purified nucleic acid segment having semiconservative or conservative amino acid changes or being a truncated segment when compared to the nucleotide sequence of SEQ ID NO:24 or 26;
(H) a purified nucleic acid segment having at least one nucleic acid segment sufficiently duplicative of the nucleic acid segment in accordance with SEQ ID NO:24 or 26 to allow possession of the biological property of encoding for a single-action of Pasteurella multocida heparin synthase;
(I) a purified nucleic acid segment encoding a modified heparin synthase, wherein the modified heparin synthase is capable of adding at least one of GlcUA and GlcNAc to a heparin polymer, and wherein the modified heparin synthase is at least about 70% identical to SEQ ID NO:2, 4, 6, 13, 15 or 34; and
(J) a purified nucleic acid segment comprising a nucleic acid sequence at least about 70% identical to SEQ ID NO:1, 3, 5, 12, 14 or 33, and wherein the purified nucleic acid segment encodes a modified heparin synthase capable of adding at least one of GlcUA and GlcNAc to a heparin polymer.

11. The purified nucleic acid segment of claim 10 wherein the purified nucleic acid segment is provided in a recombinant vector selected from the group consisting of a plasmid, cosmid, phage, integrated cassette or virus vector.

12. The purified nucleic acid segment of claim 10 wherein the recombinant vector containing the purified nucleic acid segment is used to electroporate, transform or transduce a host cell to produce a recombinant host cell having the recombinant vector.

13. The purified nucleic acid segment of claim 10 wherein the recombinant host cell produces a modified heparin synthase is capable of adding at least one of GlcUA and GlcNAc to a heparin polymer.

14. A method for enzymatically producing a polymer, comprising the steps of:
providing a functional acceptor, wherein the functional acceptor has at least two sugar units selected from the group consisting of uronic acid and hexosamine;
providing a modified heparin/heparosan synthase capable of elongating the functional acceptor, wherein the modified heparin/heparosan synthase is a single action glycosyltransferase capable of adding only one of GlcUA or GlcNAc and has an amino acid sequence encoded by the nucleic acid segment of claim 10; and
providing at least one of UDP-GlcUA, UDP-GlcNAc and UDP-sugar analogs such that the modified heparin/heparosan synthase elongates the functional acceptor in a single step manner so as to provide a polymer.

15. The method of claim 14 wherein, in the step of providing a functional acceptor, uronic acid is further defined as a uronic acid selected from the group consisting of GlcUA, IdoUA, and GalUA.

16. The method of claim 14 wherein, in the step of providing the functional acceptor, hexosamine is further defined as a hexosamine selected from the group consisting of GlcNAc, GalNAc, GlcN and GalN.

17. The method of claim 14 wherein, in the step of providing the functional acceptor, the functional acceptor has about three sugar units.

18. The method of claim 14 wherein, in the step of providing the functional acceptor, the functional acceptor has about four sugar units.
